(19) United States
(12) Patent Application Publication
(10) Pub. No.: US 2021/0230128 A1
(43) Pub. Date: Jul. 29, 2021

(54) ANTI-BLUE LIGHT COMPOUND, PREPARATION METHOD AND APPLICATION THEREOF

(71) Applicant: EUTEC NEW MATERIALS TECHNOLOGY (SUZHOU) CO., LTD., Taicang, Jiangsu (CN)

(72) Inventors: Hanmin HU, Suzhou, Jiangsu (CN); Yufeng ZENG, Taicang, Jiangsu (CN); Haitao WEI, Taicang, Jiangsu (CN); Fangyuan JIANG, Taicang, Jiangsu (CN)

(73) Assignee: EUTEC NEW MATERIALS TECHNOLOGY (SUZHOU) CO., LTD., Taicang, Jiangsu (CN)

(21) Appl. No.: 17/052,198

(22) PCT Filed: Mar. 13, 2019
(86) PCT No.: PCT/CN2019/077917

§ 371 Date: Nov. 1, 2020

(30) Foreign Application Priority Data
May 3, 2018 (CN) 201810413592.8

(51) Int. Cl. C07D 265/22; C07D 249/20; C07D 403/10; C07D 251/24; C07C 229/44; C07D 413/10; C07D 215/22; C07D 209/82; C07D 333/76; C07C 231/12; C07C 245/06; C07C 233/56; C07C 249/02

(52) U.S. Cl. CPC C07D 265/22; C07D 249/20; C07D 403/10; C07D 251/24; C07C 229/44; C07D 413/10; C07C 249/02; C07D 209/82; C07D 333/76; C07C 231/12; C07C 245/06; C07C 233/56; C07D 215/22

(57) ABSTRACT

Disclosed is a blue light absorbing compound, its preparation method and use. The compound has high stability and is suitable for high temperature processing conditions as well as outdoor application. A method of covalently bonding a blue light absorbing compound with an ultraviolet light absorbing compound for increasing its stability is also provided. The compound is capable of absorbing or blocking ultraviolet light (UVA, UVB) and blue light to protect eyes. But long-wavelength blue light can be absorbed diminishingly, so that the transmitted light has a particularly good visual experience.

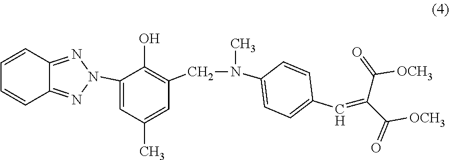 [SMILES: COC(=O)C(=Cc1ccc(N(C)Cc2cc(C)cc(-n3nc4ccccc4n3)c2O)cc1)C(=O)OC]

### FIELD

The present invention relates to blue light absorbing compound, its preparation and use. The compound possesses high stability, is suitable to high temperature processing conditions and outdoor use. The present invention also relates to a method to increase stability through covalently coupling of blue light absorbing compounds and ultraviolet light absorbing compounds.

### BACKGROUND

It is known that ultraviolet light can cause free radicals which are harmful to human body. UVA (about 320-400 nm) ultraviolet light can penetrate glass and is the main harmful ultraviolet light indoors. UVB (approximately 290-320 nm) ultraviolet light is the main ultraviolet light that causes photobiological effects of solar radiation on the skin. Short-wavelength blue light can trigger free radicals in the body, and macular degeneration will occur for a long time exposure (Investigative Ophthalmology & Visual Science (20140731), 55(7), pp. 4119-4127). In some applications, simultaneous absorption of UVA (about 320-400 nm) and full-band blue light (about 380 nm-450 nm) is required. Compounds that can absorb ultraviolet light and short-wavelength blue light are commonly used in the plastic, film or lens industries. In high-end applications, the requirement of blue light blocking agents is that as the wavelength increases, the absorption decreases gradually, that is, as the wavelength increases, the transmittance of blue light increases. In this way, it can create a better visual sense.

At present, the blue light blocking compounds are mostly inorganic compounds. Due to the poor compatibility between inorganic compounds and film resins, the light transmittance of the film is generally not good enough. (Journal of Shandong University of Science and Technology, Vol. 30(4) 2011 August p. 71-85).

In contrast, the organic compounds for blue light blocking have better compatibility with the film resin. However, they are still not stable enough and prone to degradation during high temperature processing or outdoor use. For example, in the injection processing of polycarbonate (PC) lenses, when the temperature is above 300° C., most blue light blocking organic compounds will be degraded, thus cannot be applied in high-temperature injection processes.

Therefore the requirements for being a high-end blue light blocking agent are very severe. There are very few blue light blocking products on the market that meet above requirements.

### SUMMARY

There are three requirements for a blue light blocking agent with good light transmission in the industry, which are as follows: 1. high stability or thermal processability, 2. good visual perception performance, 3. simultaneous absorption of ultraviolet light (UVA, UVB) and blue light.

The inventor designed novel polycyclic compounds to meet these three requirements at the same time. The strategy of the present invention is to covalently link the blue light absorbing groups and the ultraviolet light absorbing groups. This new design concept has achieved a surprising effect.

Surprisingly, the compounds of formula (1) of the present invention have high thermal stability, which can even be used up to 300° C. or higher. They can be applied to the injection processing of engineering plastic lenses, such as polycarbonate (PC). In addition, the compounds of formula (1) of the present invention can simultaneously absorb ultraviolet light and short-wavelength blue light. Furthermore, the compound of the present invention has a high visual perception (long-wavelength blue light can be absorbed diminishingly).

Using commercial UV or blue light blocking agent, UV-P or blue-1, as references, we compared the thermal stability with that of new compound of example 4 of present invention.

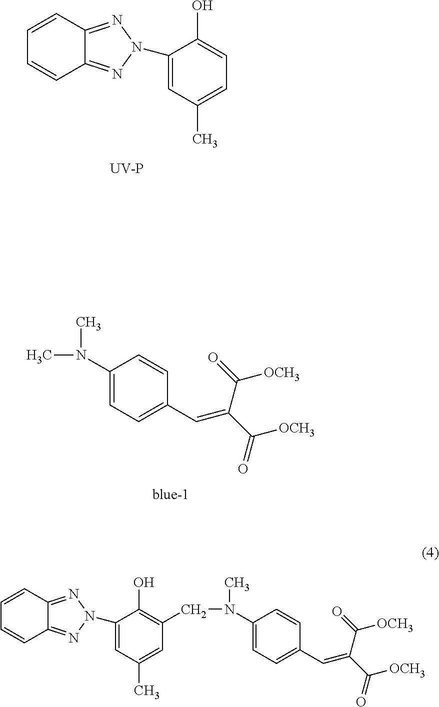 [SMILES: Cc1ccc(O)c(-n2nc3ccccc3n2)c1 ; "UV-P" ; COC(=O)C(=Cc1ccc(N(C)C)cc1)C(=O)OC ; COC(=O)C(=Cc1ccc(N(C)Cc2cc(C)cc(-n3nc4ccccc4n3)c2O)cc1)C(=O)OC]

Thermogravimetric analyzer (TGA) results showed that there is a 5% weight loss at 190° C. for UV-P, and a 5% weight loss at 178.3° C. for blue-1. Such weight losses cause UV-P and blue-1 cannot be applied on high-temperature plastic injection processing. Surprisingly, the TGA chart of FIG. 5 shows an excellent effect of the blue light-absorbing compound (4) of Example 4 of the present invention. While heating to 300° C., a thermal weight loss is less than 1%. Due to the significantly improvement of thermal stability, compound (4) is well suited for injection condition of polycarbonate (PC). It is very surprising that covalent bonding of UV-P and blue-1 cause an ultra-high thermal stability than expected.

The concept and method of the present invention that covalently couple a blue-absorbing compound and an ultraviolet-absorbing compound to increase stability has not been found or even tried.

The chemical structure of the present invention is shown in formula (1):

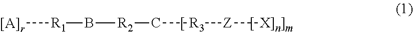

The structure of novel compounds of the present invention are polycyclic. One of the structure features is that it includes at least three rings of A, B, and C and the second feature is the blue absorbing group,

R₂—C [symbol] R₃---Z--[—X]ₙ]ₘ,

wherein, R₁˜R₃ are a bond or/and any divalent linking groups; A˜C are unsubstituted or one or more R₄ substituted benzene ring, benzo carbocyclic ring, nitrogen-containing heterocyclic ring, or benzo nitrogen-containing heterocyclic ring;

R₄ is each independently selected from hydrogen, halogen, hydroxyl, amino, nitro, cyano, linear or branched C₁˜C₁₈ alkyl, C₁˜C₁₈ alkenyl, phenyl, OR₅, SR₅, SO₂R₅, SO₃R₅, COOR₅, COR₅, OCOR₅, C(O)NR₆R₇, SO₂NR₆R₇, and NR₆R₇, wherein R₅, R₆, and R₇ are independently hydrogen, or linear or branched C₁˜C₈, preferably, R₅, R₆, and R₇ are independently hydrogen, or a linear or branched C₁-C₄ alkyl group, preferably, the halogen is selected from chlorine and bromine;

X is one or more groups, each independently selected from COOR₈, CN, CONR₆R₇ and COR₈, preferably, X is 1 or 2, each independently selected from COOR₈, CN, CONR₆R₇ and COR₈, more preferably, X is 2 groups, each independently selected from COOR₈ and CN, wherein R₈ is selected from H, linear or branched C₁˜C₁₈ alkyl, C₁˜C₁₈ alkenyl, and polyethylene glycol with molecular weight 50˜1000 daltons, preferably, R₈ is selected from H, linear or branched C₁˜C₈ alkyl;

Z is a carbon atom, Z and R₃ are connected via single, or double, or triple bond, preferably, Z and R₃ are connected via a double bond;

Z and X are connected via 1 or 2 single bonds, preferably, when n=2, Z and X are connected via 2 single bonds;

C ring and R₃ are connected via 1 or 2 single bonds, preferably, when m=1, C and R₃ are connected via 1 single bond;

[A]r and R₁ are connected via 1 or 2 single bonds, preferably, when r=1, [A]r and R1 are connected via 1 single bond;

m=1˜4, preferably, m=1˜2, particularly preferably, m=1;

n=1˜3, preferably, n=1˜2, particularly preferably, n=2;

r=1˜3, preferably, r=1˜2, particularly preferably, r=1;

particularly preferably, m=1, n=2, r=1, and its structure is as follows:

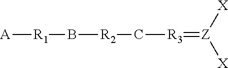

Preferably, in the compound (1) of the present invention,

R₂—C [symbol] R₃---Z--[—X]ₙ]ₘ

is a blue light absorbing group;

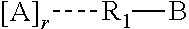

is an ultraviolet light absorbing group.

There is at least one blue light absorbing group, which is selected from:

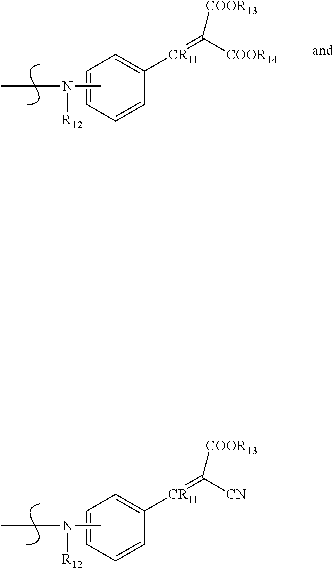

wherein, R₁₁˜R₁₄ are the same or different, and are independently selected from H, linear or branched C₁˜C₂₀ alkyl or alkenyl, and unsubstituted or substituted phenyl, preferably, R₁₁˜R₁₄ are H, linear or branched C₁˜Cᵢₛ alkyl or alkenyl, unsubstituted or halogen or C₁˜C₆ substituted phenyl, more preferably, R₁₁˜R₁₂ are H, linear or branched C₁˜C₈ alkyl, unsubstituted or substituted with C₁˜C₄ phenyl, R₁₃˜R₁₄ are H, linear or branched C₁˜C₈ alkyl or alkenyl, unsubstituted or substituted by halogen or C₁˜C₄ phenyl, and,

there is at least one ultraviolet light absorbing group, which is selected from:

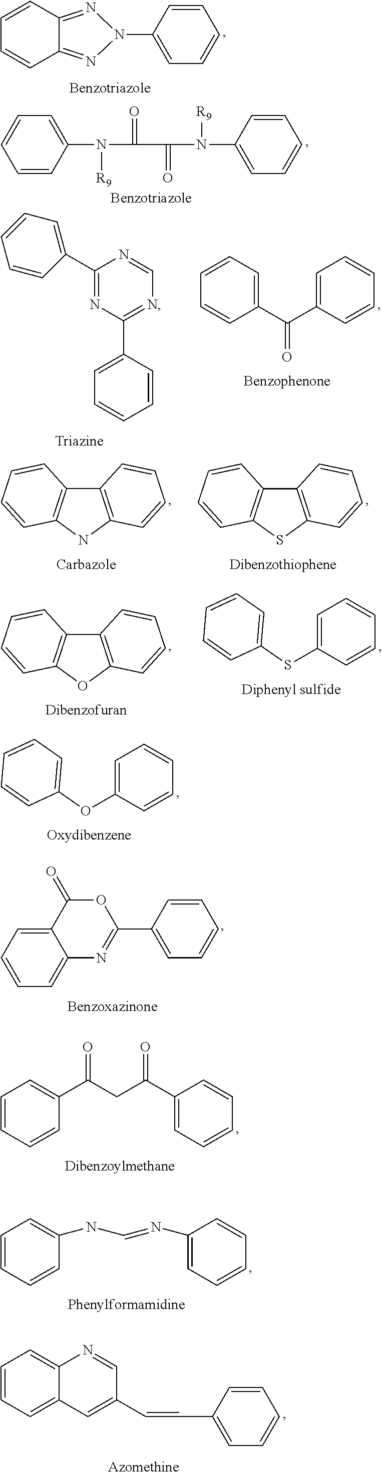

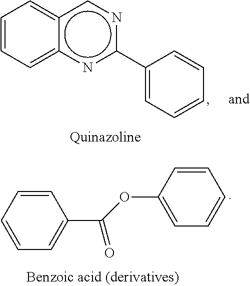

Preferably, in the compound (1) of the present invention, R₁˜R₃ are a single bond or/and any divalent linking group, preferably, R₁˜R₃ are a bond or/and a chain composed of 1-10 selected from the following groups: —O—, —S—, —C(═O)—, —COO—, —C(═S)—, —C(═NR₉)—, —CH₂—, —CH(R₉)—, —C(R₉)₂—, —C(R₉)═, —C≡, —C(R₉)═C(R₉)—, —C≡C—, —N(R₉)—, —C(R₉)═N—, phenyl, more preferably, R₁˜R₃ are a bond or/and a chain composed of 1˜6 groups selected from the following: —O—, —S—, —C(═O)—, —COO—, —C(═NR₉)—, —CH₂—, —CH(R₉)—, —C(R₉)₂—, —C(R₉)═, —C(R₉)═C(R₉)—, —N(R₉)—, —C(R₉)═N—, phenyl, preferably, R₁ and R₃ are a bond or/and a chain composed of any of —(R₉)N—CH═N—, —NH—C(═O)—C(═O)—NH—, —COO—, —CON—, —CH₂CH₂CON—, —CH═N—, —(CHR₉)qN(R₉)—, preferably, R₂ is a bond, or —(CHR₉)qN(R₉)—, particularly preferably, R₂ is a bond, —CH₂N(CH3)-, or —CH₂N(CH₂CH₃)—, q=0˜8, preferably, q=0˜4, more preferably, q=1˜2, particularly preferably, q=1. R₉ is H, linear or branched C₁˜C₈ alkyl, unsubstituted phenyl, or phenyl substituted by OH, halogen, C₁˜C₄ alkoxy, linear or branched C₁˜C₄ alkyl, more preferably, R₉ is H, linear or branched C₁˜C₄ alkyl, or unsubstituted phenyl, more preferably, R₉ is H, linear or branched C₁˜C₂ alkyl.

Preferably, in the compound (1) of the present invention,

A is selected from:

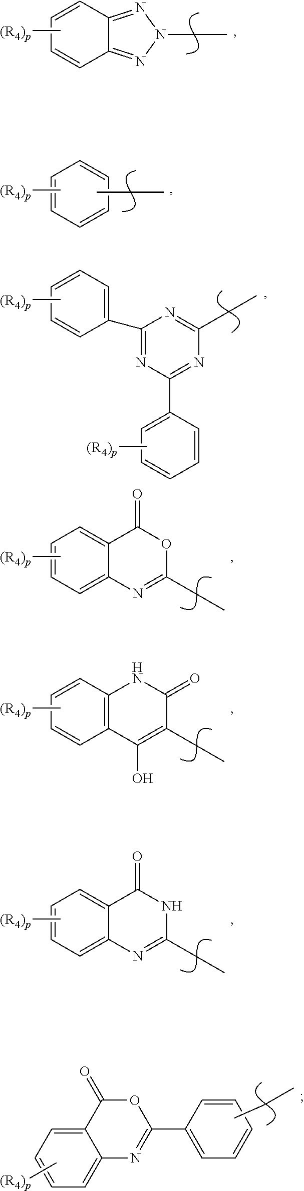

B is selected from:

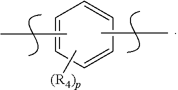

C is selected from:

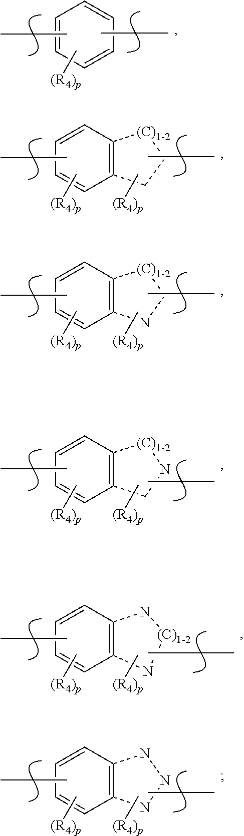

R₄ is one or more substituents, and each independently selected from hydrogen, halogen, nitro, cyano, linear or branched C₁˜C₈ alkyl, C₁˜C₈ alkenyl, OR₅, SR₅, SO₂R₅, COOR₅, COR₅, C(O)NR₆R₇, NR₆R₇,

and the R₄ can form a 3-6 atom fused ring, wherein R₅, R₆, and R₇ are independently hydrogen, or a linear or branched C₁˜C₈ alkyl group; p=1˜3, preferably, p=1˜2.

Preferably, for

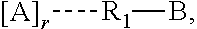

r is 1 or 2. when r is 2, ----R₁— is a bond, and B is triazine,

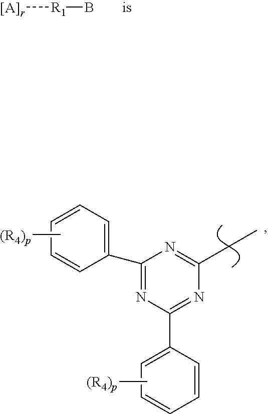

when r=1, ----R₁ is a bond or a chain, and B is a ring,

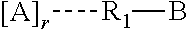

is selected from:

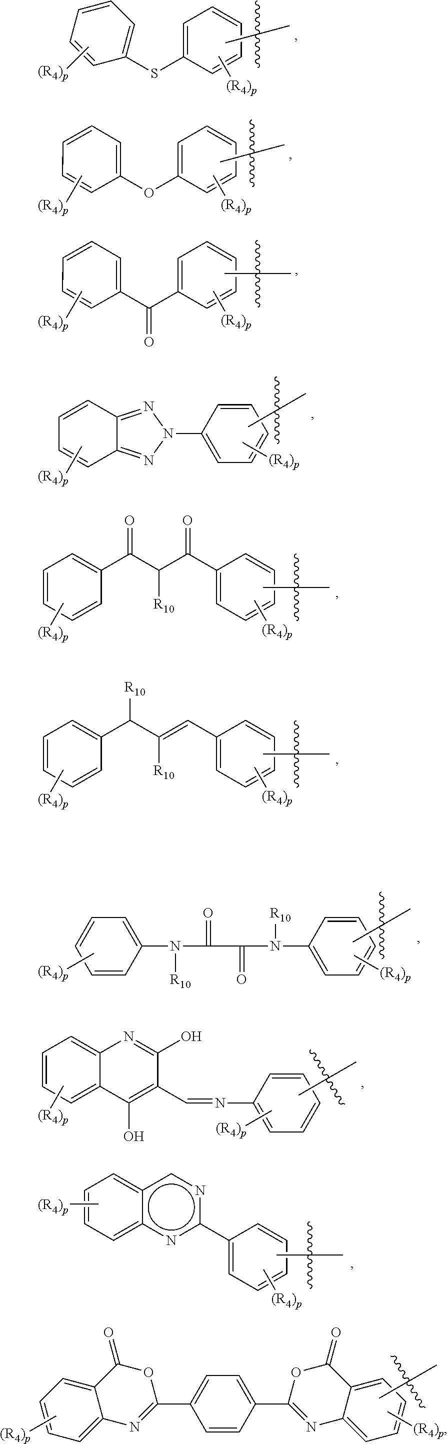

when r=1, ----R₁ is a bond and a chain, and B is a ring,

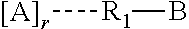

is selected from:

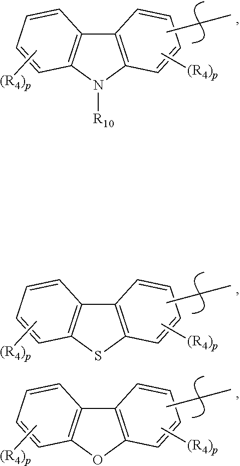

wherein, R₄ is one or more substituents, and each is independently selected from hydrogen, halogen, nitro, cyano, linear or branched C₁˜C₈ alkyl, C₁˜C₈ alkenyl, OR₅, SR₅, SO₂R₅, COOR₅, COR₅, C(O)NR₆R₇, NR₆R₇, wherein, R₅, R₆, and R₇ are independently hydrogen, or linear or branched C₁˜C₆ alkyl;

p=1˜3, preferably, p=1˜2; R₁₀ is H, linear or branched C₁˜C₈ alkyl, phenyl or substituted phenyl, preferably, R₁₀ is H, linear or branched C₁˜C₆ alkyl, or a phenyl.

More preferably, compound (1) of the present invention has a polycyclic structure, characterized in that B═C=benzene ring, and Z and R₃ are connected via a double bond, and its structure is as shown in the compound of formula (2),

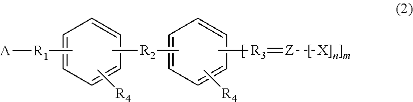

wherein, R₁ and R₃ are a bond or a chain consisting of 1-6 of the following groups: —O—, —N(R₉)—, —C(═O)—, —COO—, —CH2-, —CH(R₉)—, —C(R₉)₂—, —C(R₉)═, —C≡, —C(R₉)═N—, phenyl, preferably, R₁ and R₃ are a bond or/and —(R₉)N—CH═N—, —NH—C(═O)—C(═O)—NH—, —COO—, —CON—, —CH₂CH₂CON—, or —CH═N—; R₂ is a bond, or —(CHR₉)qN(R₉)—, preferably, R₂ is a bond, or —CH2N(R₉)—, more preferably, R₂ is a bond, —CH₂N(CH₃)—, or —CH₂N(CH₂CH₃)—; R₄ is one or more substituents, and each is independently selected from hydrogen, halogen, nitro, cyano, straight or branched C₁˜C₈ alkyl, C₁˜C₈ alkenyl, OR₅, SR₅, SO₂R₅, COOR₅, COR₅, C(O)NR₆R₇, NR₆R₇, wherein R₅, R₆, and R₇ are independently hydrogen, or a linear or branched C₁˜C₆ alkyl group, preferably, R₅, R₆, and R₇ are independently hydrogen, or linear or branched C₁˜C₄ alkyl group, multiple R₄ can form a fused ring with the benzene ring; R₉ is H, linear or branched C₁˜C₈ alkyl, unsubstituted phenyl, or phenyl substituted by OH, halogen, C₁˜C₄ alkoxy, linear or branched C₁˜C₄ alkyl, more preferably, R₉ is H, linear or branched C₁˜C₄ alkyl, or unsubstituted phenyl, particular preferably, R₉ is H, linear or branched C₁˜C₂ alkyl;

X is one or more, each independently selected from COOR₈, CN, CONR₆R₇ and COR₈, preferably, X is one or two, each independently selected from COOR₈, CN;

Z is a carbon atom, and Z and X are connected via a single or double bond, preferably, Z and X are connected via a single bond;

m=1˜2, preferably, m=1; n=1˜2, preferably, n=2; q=0˜8, preferably, q=0˜4, more preferably, q=1˜2, particularly preferably, q=1.

Particular preferably, The compound (1) of the present invention, has a polycyclic structure, characterized in that B═C=benzene ring, and Z and R₃ are connected via a double bond, and its structure is as follows:

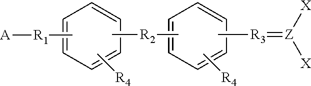

wherein, X is the same or different, each independently selected from COOR₈, CN, CONR₆R₇ and COR₈, preferably, X is the same or different, each independently selected from COOR₈, CN;

Z is a carbon atom;

A is selected from:

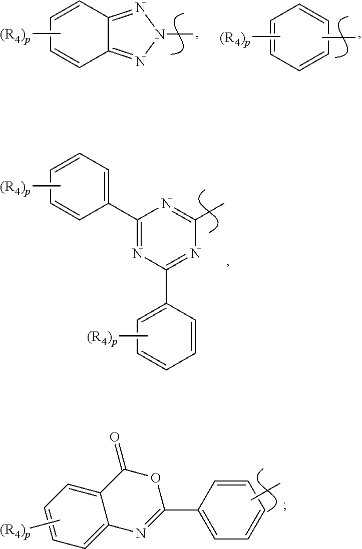

R₁ and R₃ are a bond or a chain consisting of 1-6 of following groups: —O—, —N(R₉)—, —C(═O)—, —COO—, —CH2-, —CH(R₉)—, —C(R₉)₂—, —C(R₉)═, —C(R₉)═N—, phenyl, preferably, R₁ and R₃ are a bond or/and —(R₉)N—CH═N—, —NH—C(═O)—C(═O)—NH—, —COO—, —CON—, —CH₂CH₂CON—, —CH═N—;

R₂ is a bond, or —(CHR₉)qN(R₉)—, preferably, R₂ is a bond, or —CH₂N(R₉)—, more preferably, R₂ is a bond, —CH₂N(CH₃)—, or —CH₂N(CH₂CH₃)—; q=0˜8, preferably, q=0˜4, more preferably, q=1˜2, particularly preferably, q=1.

R₄ is one or more substituents, and each is independently selected from hydrogen, halogen, nitro, cyano, linear or branched C₁˜C₈ alkyl, C₁˜C₈ alkenyl, OR₅, SR₅, SO₂R₅, COOR₅, COR₅, C(O)NR₆R₇, NR₆R₇, wherein R₅, R₆, and R₇ are independently hydrogen or a linear or branched C₁˜C₆ alkyl, preferably, R₅, R₆, and R₇ are independently hydrogen, or linear or branched C₁˜C₄ alkyl group, multiple R₄ can form a fused ring with a benzene ring; R₉ is H, linear or branched C₁˜C₈ alkyl, unsubstituted phenyl, or phenyl substituted by OH, halogen, C₁˜C₄ alkoxy, linear or branched C₁˜C₄ alkyl, more preferably, R₉ is H, linear or branched C₁˜C₄ alkyl, or unsubstituted phenyl, particularly preferably, R₉ is H, linear or branched C₁˜C₄ alkyl.

Particularly preferably, the compound (1) of the present invention, is as follows:

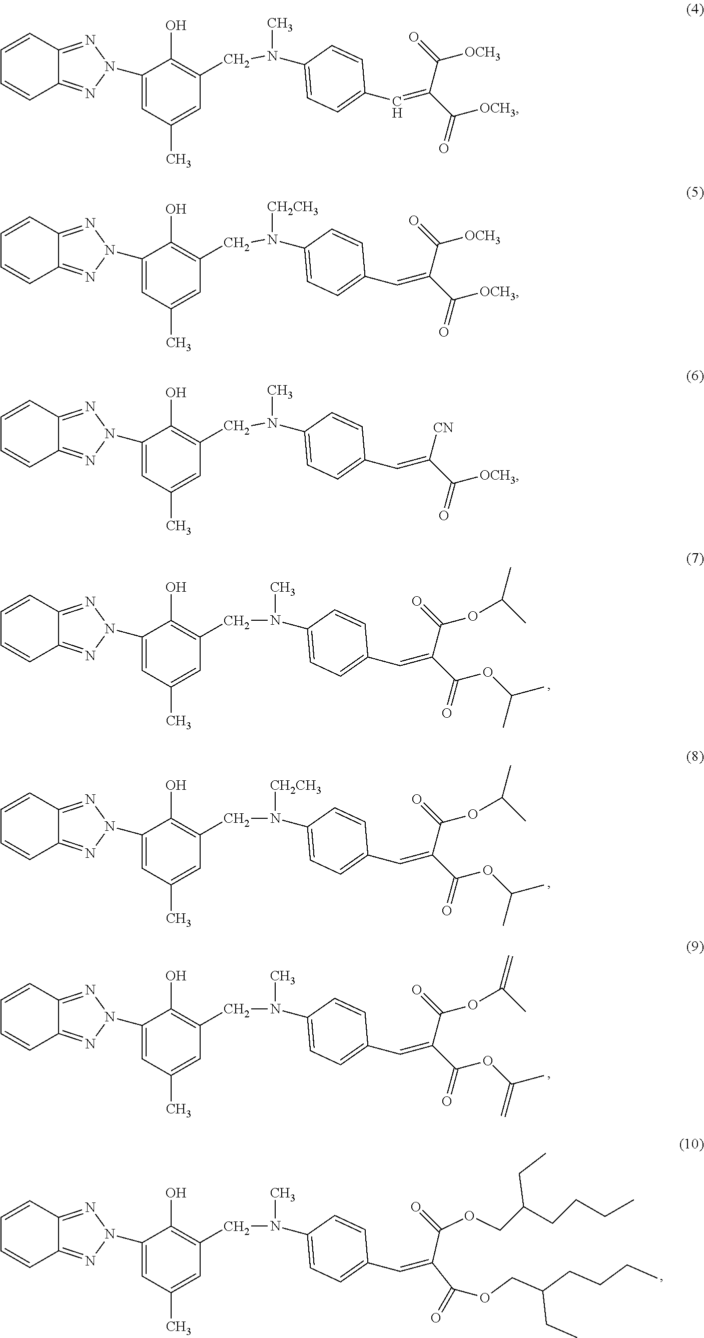

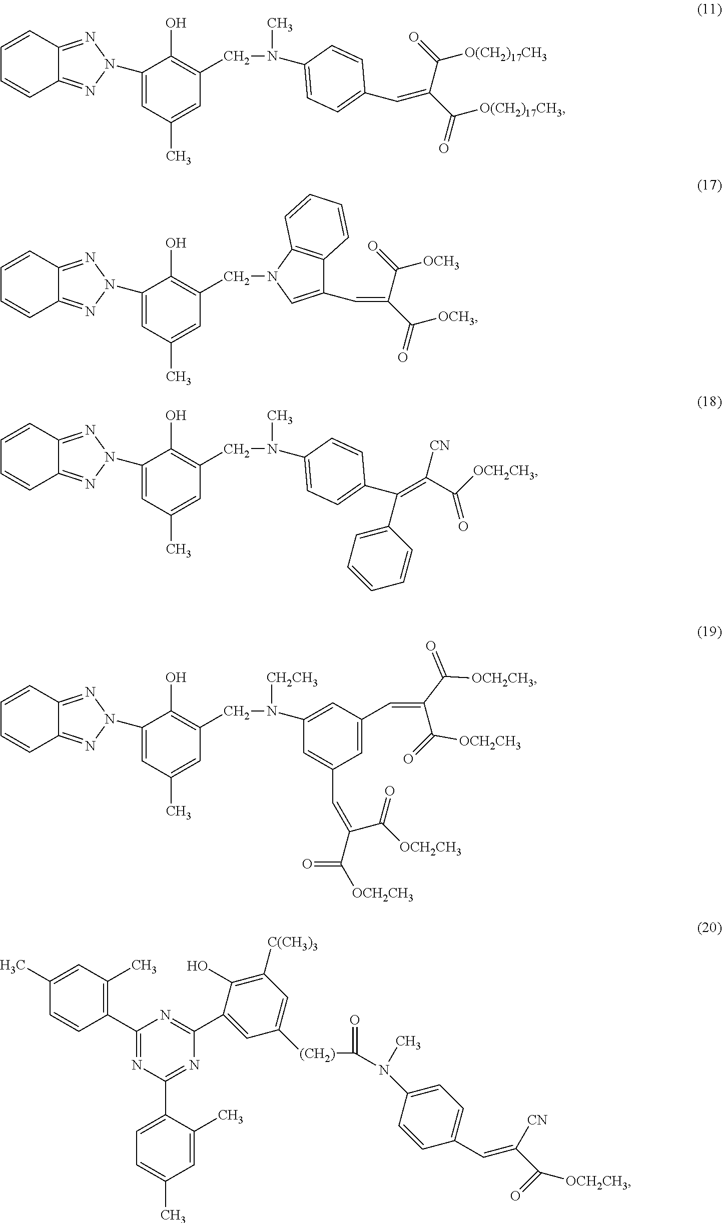

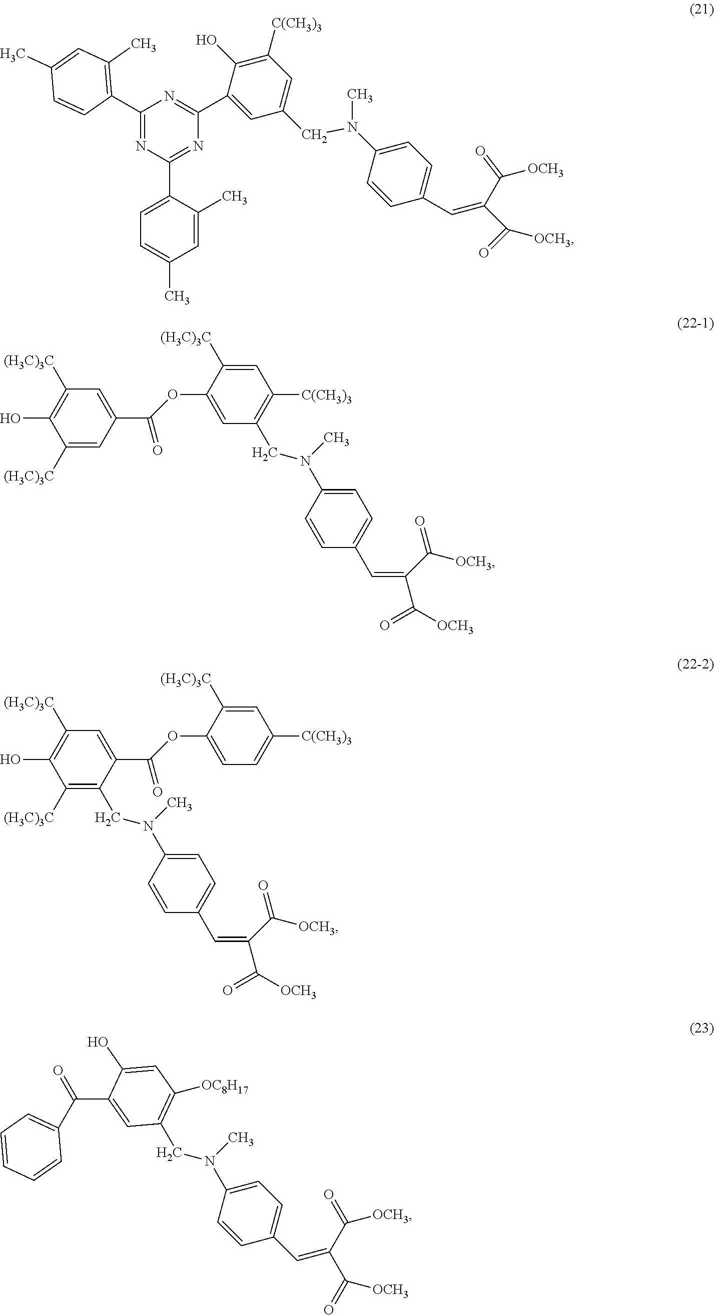

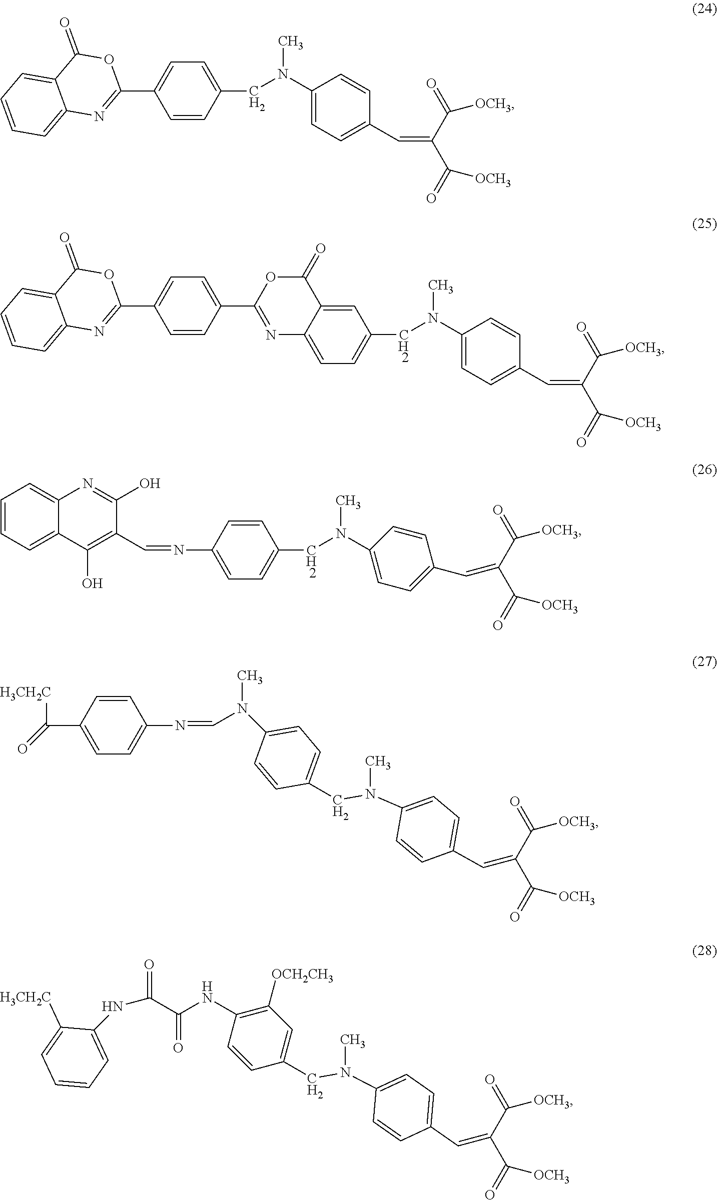

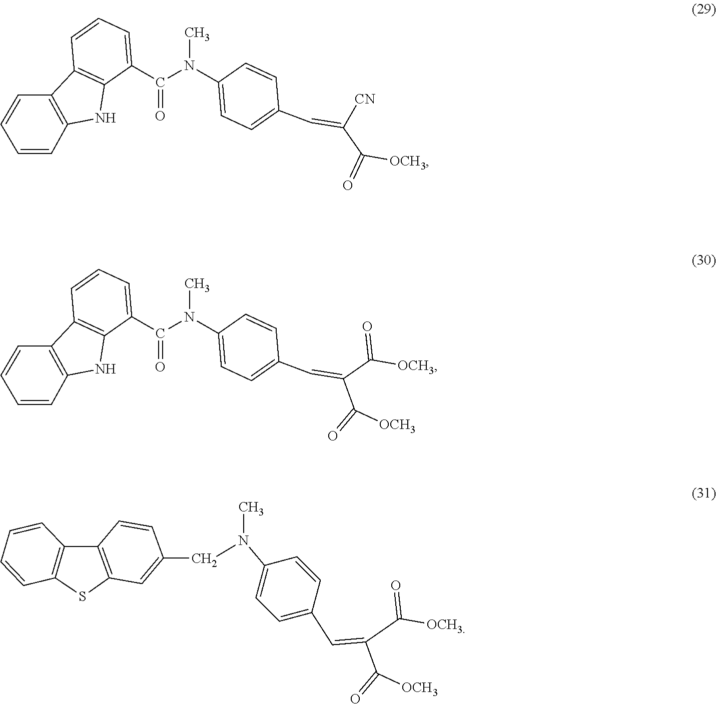 [SMILES: COC(=O)/C(C#N)=C/c1ccc(N(C)C(=O)c2cccc3c2[nH]c2ccccc23)cc1 ; COC(=O)C(=Cc1ccc(N(C)C(=O)c2cccc3c2[nH]c2ccccc23)cc1)C(=O)OC ; COC(=O)C(=Cc1ccc(N(C)Cc2ccc3c(c2)sc2ccccc23)cc1)C(=O)OC]

The present invention also relates to a method for producing a high-stability blue light blocking compound, which is characterized in that the blue light and the ultraviolet light blocking compound are covalently bonded.

The preparation method of the compound of formula (1) of the present invention includes the following reaction steps:

A-R₁—B—R₂—C—CHO+Z--[—X]ₙ→formula (1) compound

or

A-R₁—B—CH₂Cl+NHR₁₅—C [symbol] -R₃---Z--[—X]ₙ]ₘ→formula(1)compound

R₁₅ is H, linear or branched C₁˜C₈ alkyl or phenyl; A˜C, C₁˜C₈, X, Z are as defined in compound (1).

The first method of the above can be seen in example 1˜18, and the second method can be seen in example 19-31.

### Example 1-18

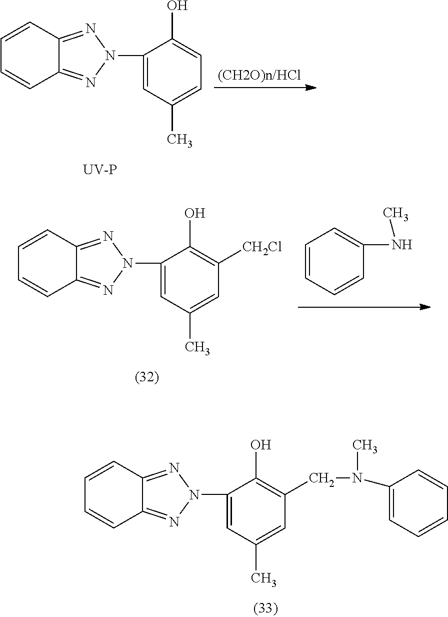

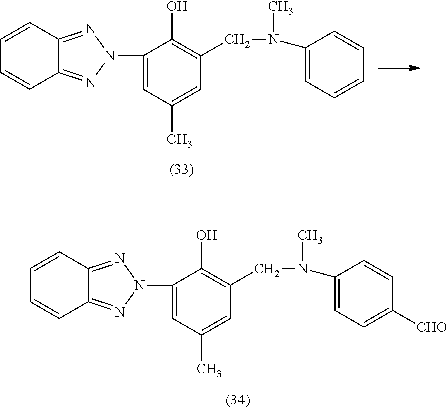 [SMILES: Cc1cc(CN(C)c2ccccc2)c(O)c(-n2nc3ccccc3n2)c1 ; Cc1cc(CN(C)c2ccc(C=O)cc2)c(O)c(-n2nc3ccccc3n2)c1]

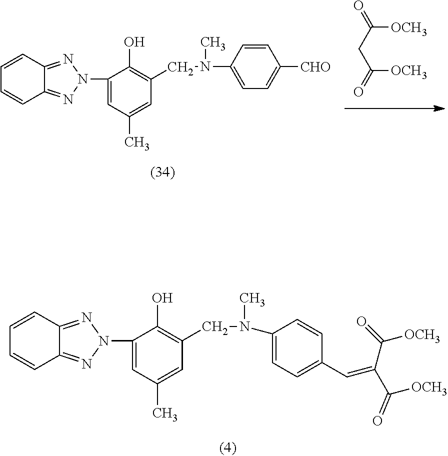 [SMILES: Cc1cc(CN(C)c2ccc(C=O)cc2)c(O)c(-n2nc3ccccc3n2)c1 ; COC(=O)CC(=O)OC ; COC(=O)C(=Cc1ccc(N(C)Cc2cc(C)cc(-n3nc4ccccc4n3)c2O)cc1)C(=O)OC]

Method 2:

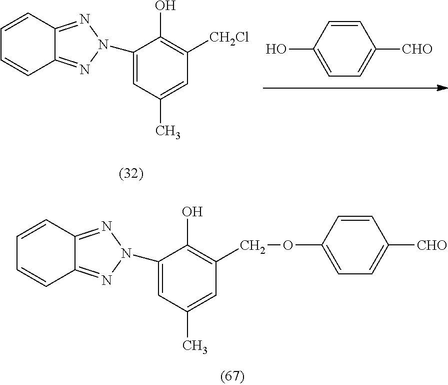

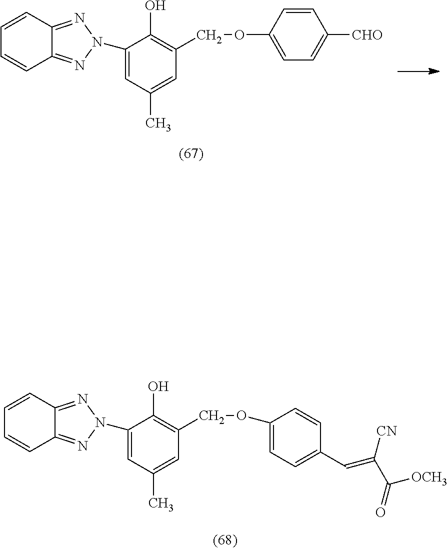 [SMILES: Cc1cc(COc2ccc(C=O)cc2)c(O)c(-n2nc3ccccc3n2)c1 ; COC(=O)/C(C#N)=C/c1ccc(OCc2cc(C)cc(-n3nc4ccccc4n3)c2O)cc1]

P-Hydroxybenzaldehyde, potassium carbonate and compound (32) were mixed to react overnight in DMF at 80° C. under nitrogen protection to obtain compound (67). 2-cyano ethyl acetate was added to the mixture to obtain compound (68).

Method 3:

The Preparation Method of Compound (17) (Example 17):

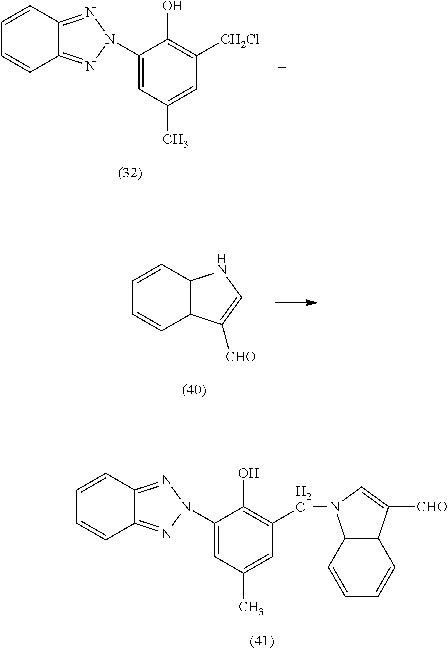

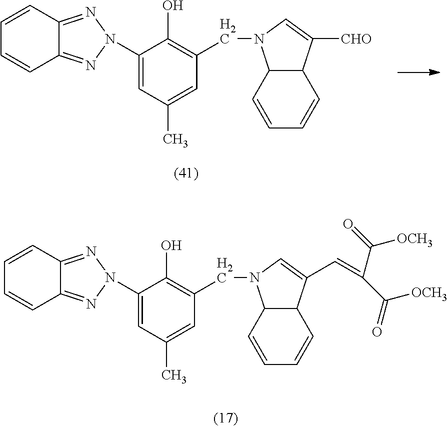 [SMILES: Cc1cc(CN2C=C(C=O)C3C=CC=CC32)c(O)c(-n2nc3ccccc3n2)c1 ; COC(=O)C(=CC1=CN(Cc2cc(C)cc(-n3nc4ccccc4n3)c2O)C2C=CC=CC12)C(=O)OC]

Method 4:

The Preparation Method of Traditional Benzotriazole Compound (Example 13):

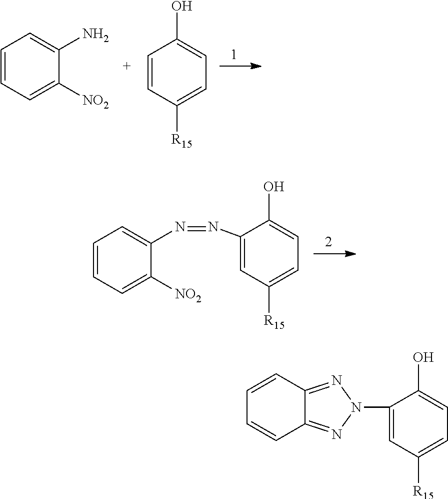

The synthetic method usually starts with the reaction between 2-nitro-aniline and various substituted phenols. The first step is to form an azo compound, and the second step is a reduction reaction to form a benzotriazole compound (U.S. Pat. No. 3,773,751). R₁₅ is selected from halogen, hydroxyl, amino, nitro, cyano, linear or branched C₁˜C₁₈ alkyl, C₁˜C₁₈ alkenyl, phenyl, OR₅, SR₅, SO₂R₅, SO₃R₅, COOR₅, COR₅, OCOR₅, C(O)NR₆R₇, SO₂NR₆R₇, and NR₆R₇, wherein R₅, R₆, and R₇ are independently hydrogen or a linear or branched C₁˜C₁₈ alkyl group.

Method 5 (Example 19-31):

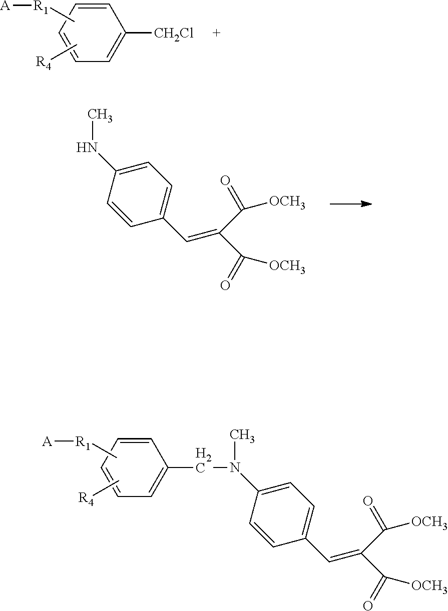

The present invention also relates to a blue light or/and ultraviolet light blocking composition, which is characterized in that it comprises a compound structure of formula (1), and the composition can be used to manufacture optical films, optical lenses, goggles, coatings, adhesives, or panels and other products.

The present invention also includes blue light or/and ultraviolet light blocking lenses or goggles, made by glass and polymer lenses, such as polycarbonate (PC), polymethylmethacrylate (PMMA), nylon (PA), TPX (Polymethylpentene), polystyrene, diethylene glycol dialkyl carbonate resin (PEDC). The blue light blocking agents can be added to the resin in a specific ratio for co-molding, and the content of the blue light agent is 0.01%˜20%, preferably 0.05%˜10%, more preferably 0.1%˜5%. In the present invention, an impregnation process can also be adopted to impregnate the lens in a composition containing blue light blocking agent. The invention can also adopt a thin film process to form a blue light blocking film on the surface of the lens. The present invention can also adopt a transfer coating process, for example, first coat the pre-mix on a release film, and then transfer to the optical lens. Generally, about 1 to 5% of the compound of the example is added to a 50 μm film.

The basic structure of the blue or/and ultraviolet light blocking system of the present invention includes one or more blue light blocking layers, and/or a base layer, and/or a release layer. Basically, the blue light blocking composition is coated on the base layer or release layer and then dried. Or adopt a transfer coating process, which is first coated on the release film and then transferred to the base layer. A layer of release film is attached to the upper and lower layers of the blue light blocking film and is called OCA (Optically Clear Adhesive). Coating methods are known techniques, including traditional brush coating, spray coating, curtain coating, roll coating, slit coating, air knife coating, knife coating, and metering rod coating. Drying methods include natural drying, microwave drying, ultraviolet drying, infrared drying, and hot air drying. The base layer includes one or a mixture of polyester, glass, polyethylene, polypropylene, polycarbonate, polyamide, polyacrylate, polymethacrylate, polyvinyl acetate, and polyvinyl chloride. The release film includes silica-based and non-silicone-based materials. Non-silicone compounds include, for example, one or more mixtures of polyethylene, polypropylene, polyurea, polyacrylic acid, polyester, and fluorocarbon. OCA optical glue can be used in different fields according to the thickness, such as transparent device bonding, display assembly, lens assembly, panel, glass or polycarbonate and other plastic materials. The blue light blocking film may also include other film layers, for example, a UV absorbing film layer, an anti-fog film layer, and an antistatic film layer. The blue light blocking film can be used in the optical or electronic industries, such as optical lenses, goggles, lenses, displays, panels, and lighting protection.

The ultraviolet or blue light blocking agent, with various functional additives, and polyvinyl chloride, low-density polyethylene (LDPE), ethylene-vinyl acetate copolymer (EVA), metallocene linear polyethylene (MLLDPE) and other resin materials can make the film through blow molding and calendering process. A manufacturing example includes mixing 1 kg of low-density polyethylene as base material, 200 g of metallocene linear polyethylene, 5 g of blue light blocking agent, 8 g of antioxidant, 5 g of ultraviolet absorber, and 9 g of glyceride, and passing them through conventional blow molding. Finally, a transparent film with a thickness of 0.03˜0.50 mm can be obtained.

The added amount of the blue light blocking agent of the present invention depends on the type and thickness of the film and the required penetration ratio. Generally, it can be roughly calculated based on the standard absorption value (lcm optical path) and the actual film thickness in order to achieve penetration ratio. The range includes but is not limited to 0.001%˜20%.

The thermally induced blue light blocking composition usually includes a blue light blocking agent, thermal initiator, monomer, solvent, and auxiliary agent. Thermal initiators are classified by temperature. High temperature (above 100° C.) initiators are classified according to their working temperature range, such as one or more mixtures of alkyl peroxides, alkyl hydrogen peroxide compounds, and peroxy ester compounds. Medium temperature (40˜100° C.) initiators are also classified, such as azo compounds, dioxygen peroxide, persulfate, etc. Low temperature (0-40° C.) initiators are also classified, such as redox initiation system. Thermal initiators can be divided into azo compounds and peroxides according to their molecular structure. Commonly used azo compounds include azobisisobutyronitrile (ABIN), azobisisoheptonitrile (ABVN), and azo compounds with carboxyl or sulfonic acid groups. Commonly used peroxides include benzyl peroxide (BPO), dihydroperoxide (2,4-dichlorobenzyl), diethyl peroxide, dioctyl peroxide, dilaurin peroxide, diisopropylene peroxide (DCP), di-tert-butyl peroxide (DTBP), tert-butyl peroxybenzoate (BPB), cumene hydroperoxide (CHP) and tert-butyl hydroperoxide (TBH), Diisopropyl Peroxydicarbonate (IPP), Diisobutyl Peroxydicarbonate (IBP), peroxydicarbonate, methyl ethyl ketone peroxide, cyclohexanone peroxide, persulfate and hydrogen peroxide. Monomers are small molecular compounds containing double bonds or other active functional groups. Double bond monomers include acrylics, acrylics, methacryls, methacrylates, hydroxyacrylates, methacrylates, diacetone acrylamides, vinyls, styrenes, dienes, vinyl fluoride, vinyl chloride, acrylonitrile, vinyl acetate, silicone acrylate, epoxy acrylate, urethane acrylate. Monomers of acrylic or acrylate ester, include acrylate soft monomers, acrylate hard monomers, acrylic functional monomers, and crosslinking monomers. The preferred soft acrylate monomers are, for example, ethyl acrylate, butyl acrylate, 2-ethylhexyl acrylate, and isooctyl acrylate. The preferred acrylic hard monomers are, for example, methyl acrylate and methyl methacrylate. The preferred acrylic functional monomers are, for example, acrylic acid and methacrylic acid. The preferred crosslinking monomers are, for example, hydroxyethyl acrylate, hydroxypropyl acrylate, hydroxyethyl methacrylate, hydroxypropyl methacrylate, and dihydrazine adipate (ADH). Thermosetting resins used in blue light blocking films are, for example, polyurethane resins, epoxy resins, phenolic resins, polyurea resins, unsaturated polyester resins, and alkyd resins. The monomers can be isocyanates, epichlorohydrin, phenols, aldehydes, polyols, fatty acids, polyacids, acid anhydrides, polythiols, polyamines, alcohol amines, and thiolamines. Solvents include acetonitrile, acetone, methyl ethyl ketone, butanone, cyclohexanone, benzene, toluene, xylene, ethyl acetate, butyl acetate, methyl isobutyl ketone, methanol, ethanol, isopropanol, butanol, ethylene glycol, propylene glycol, butylene glycol, vinyl chloride, methylene chloride, chloroform, carbon disulfide, tetrahydrofuran, dimethylformamide (DMF), polyethylene glycol methyl ether (EGMME). Polyurethane is produced by the reaction of polyester polyol or polyether polyol and isocyanate. Specific embodiments, for example, polyol and isocyanate, chain extender, and catalyst (such as dimethylaminocyclohexane) are mixed, and then injected into a mold for curing. Or, the isocyanate reacts with the polyol to form a prepolymer, and then the chain extender is added. The epoxy resin monomer is produced by the reaction of epichlorohydrin and bisphenol A compounds. The specific embodiment includes reacting bisphenol A and epichlorohydrin, and then adding a hardener, such as dicyandiamide (Dicy) or dihydrazine adipic acid (ADH), and the accelerator, 2-methylimidazole. The monomers of alkyd resins include polyols and fatty acids.

Specific embodiments, for example, put glycerin, isophthalic anhydride and fatty acid into a reactor, and heat to 200-250° C. until the desired viscosity and acid value. The unsaturated polyester resin is a linear polymer compound having an ester bond and an unsaturated double bond. Monomers include unsaturated dibasic acids and unsaturated diols, or saturated dibasic acids and unsaturated diols. Specific embodiments, for example, propylene glycol, butenedioic anhydride, and phthalic anhydride are subjected to condensation polymerization in a reactor. The resulting unsaturated polyester is added with styrene monomer to become a viscous resin, and cyclohexanone peroxide is added during use. Additives can include stabilizers, coupling agents, leveling agents, defoamers, dispersants, solvents, chain transfer agents, catalysts, tougheners, tackifiers, plasticizers, thickeners, diluents, flame retardants Mixing of one or more of agents, polymerization inhibitors, preservatives, hardeners, and acid base blending agents. Chain transfer agents are used, such as aliphatic mercaptans and dodecyl mercaptan. Stabilizers are used, such as UV absorbers, hindered amines, antioxidants, anti-hydrolysis agents, peroxide traps, free radical traps.

The thermally-initiated blue light blocking composition may include an anti-blue light agent with a content of 0.01%˜20%, an initiator quality content of 0.01˜10%, a monomer or prepolymer or polymer content of 50˜99.98%, and an auxiliary agent quality The content is 0 to 80%. The content of the blue light blocking agent is preferably 0.05% to 10%, more preferably 0.1% to 5%. Specific embodiments, for example, mix acrylic soft monomers, acrylic hard monomers, acrylic functional monomers, and acrylic crosslinking monomers to form a monomer mixture. Add the monomer mixture, initiator and solvent into the reaction kettle and raise the temperature. After the reaction is completed, the temperature is reduced to room temperature, the material is discharged, and the substrate layer is evenly coated.

The photo-initiated blue blocking light composition usually includes an blue light blocking agent, polymerized monomer or/and prepolymer, photoinitiator, and auxiliary agent. Photoinitiators mainly include free radical photoinitiators and cationic photoinitiators. The free radical photoinitiators are divided into cleavage photoinitiators and hydrogen abstraction photoinitiators. The cleavage free radical type photoinitiator is mainly based on aryl alkyl ketone compounds, including benzoin derivatives, dialkoxy acetophenone, α-hydroxyalkyl phenone, α-aminoalkyl phenone, acyl phosphine oxide, esterified oxime ketone compound, aryl peroxy ester compound, halogenated methyl aromatic ketone, organic sulfur compound, benzoyl formate or the mixture. The hydrogen abstraction type free radical photoinitiator includes active amine, benzophenone, thioxanthone and its derivatives, anthraquinone, active amine, coumarone, camphorquinone or the mixture. Cationic photoinitiator, including diazonium salt, diaryliodonium salt, triarylsulfonium salt, alkylsulfonium salt, iron arene salt, sulfonyloxyketone and triarylsiloxy, or the mixture. The prepolymer can be an oligomer that contains functional groups with reactivity, such as methacrylate oligomer, acrylate oligomer, epoxy acrylate oligomer, urethane acrylate oligomer, silicone acrylate oligomer, amino acrylate oligomer, carboxyl acrylate oligomer, phosphate acrylate oligomer, hydroxy polyacrylate oligomer, polyester acrylate oligomer, polyether acrylate oligomer, or the mixture. Polymerization monomers can be one or a mixture of various small molecules for addition or condensation polymerization. Among them, double bond monomers include acrylic, acrylate, methacrylic, methacrylate, hydroxyacrylate, methacrylate, diacetone acrylamide, vinyl, styrene, dienes, vinyl fluoride, vinyl chloride, acrylonitrile, vinyl acetate, silicone acrylate, epoxy acrylate, urethane acrylate. Acrylic or acrylate monomers, including acrylate soft monomers, acrylate hard monomers, acrylic functional monomers, and crosslinking monomers. The preferred soft acrylate monomers are, for example, ethyl acrylate, butyl acrylate, 2-ethylhexyl acrylate, and isooctyl acrylate. The preferred acrylic hard monomers are, for example, methyl acrylate and methyl methacrylate. The preferred acrylic functional monomers are, for example, acrylic acid and methacrylic acid. Preferred crosslinking monomers are, for example, hydroxyethyl acrylate, hydroxypropyl acrylate, hydroxyethyl methacrylate, hydroxypropyl methacrylate, and adipic acid dihydrazide. Wherein, the content of the ultraviolet or/and blue light blocking agent of formula (1) is 0.01%˜20%, preferably 0.05%˜10%, more preferably 0.1%˜5%. Additives can include stabilizers, coupling agents, leveling agents, defoamers, dispersants, solvents, chain transfer agents, catalysts, toughening agents, tackifiers, plasticizers, thickeners, diluents, flame retardants, polymerization inhibitors, preservatives, hardeners, and acid base blending agents, or the mixture commonly used auxiliary agents include coupling agents, such as silane coupling agents. Commonly used auxiliary agents include stabilizers, such as one or more of UV absorbers, hindered amines, antioxidants, and free radical scavengers. The photo-initiated blue light blocking composition includes 0.01%-20% of blue light blocking agent, 0.01˜10% of initiator, 5˜99.98% monomer and/or prepolymer, and 0˜95% of auxiliary agent. Preferably, the content of the blue light blocking agent is 0.05%˜10%, the content of the initiator is 0.05˜5%, the monomer and/or prepolymer content is 5˜99.9%, and the content of the auxiliary agent is 0˜50%. Specific embodiments for the light-initiated blue light blocking composition (for example, a combination of acrylate monomer, acrylate prepolymer, blue light absorber, initiator Photocure 84) can be mixed and uniformly applied to a clean base layer, and the blue light blocking film is obtained by curing with UV light. A non-reactive blue light blocking composition includes an blue light blocking agent, polymer, solvent, and/or additives. It mainly uses the volatilization solvents or other dispersion media in the coating to form a solid film. The polymer can be selected from but not limited to polyacrylate, polymethacrylate, polyethylene, polypropylene, polyvinyl chloride, polystyrene, polyacrylonitrile, polyethylene terephthalate, polyethylene terephthalate butanediol, polycarbonate, polyamide, ethylene-vinyl acetate copolymer, polyvinyl alcohol, acrylonitrile-styrene copolymer, thermoplastic polyurethane, polyimide, cellulose, polysulfide benzene, polyoxyxylene, polyoxymethylene, polysulfone, polyether ether ketone, polyamide-imine, polyether imine, polyether sulfone, polyether imide, or the mixture. The non-initiating blue light blocking composition may include an blue light blocking agent having a content of 0.01%˜20%, a polymer content of 5˜99.99%, and an auxiliary agent having a content of 0˜95%. Specific embodiments, for example, the polystyrene plastic is dried and pulverized into small pieces, then poured into a xylene/ethyl acetate mixed solvent, and stirred until completely dissolved. Then add plasticizer (for example, dibutyl phthalate) and blue light block agent, heat and stir to obtain the composition. After coating and drying for removing the solvent, an blue light blocking film is obtained. Polystyrene plasticizers include phthalates, diterpenes, epoxy soybean oil, epoxy soybean oleate octyl, and alkylbenzene sulfonate.

### BRIEF DESCRIPTION OF THE DRAWINGS

FIG. 1 is a UV-VIS absorption graph of the compound L-486 (10 mg/chloroform), of the present invention;

FIG. 2 is a UV-VIS absorption graph of compound L-500 (10 mg/chloroform);

FIG. 3 is a UV-VIS absorption graph of compound L-467 (10 mg/chloroform);

FIG. 4 is a UV-VIS absorption profile of the compound L-538 (10 mg/chloroform);

FIG. 5 is a TGA diagram of compound L-486;

FIG. 6 is a TGA chart of compound L-487.

### DETAILED DESCRIPTION

The specific embodiments of the present invention are described below, but are not limited to these embodiments.

### Example 1, Preparation of 2-(2-hydroxy-3-(chloromethyl)-5-methyl)benzotriazole (Compound 32)

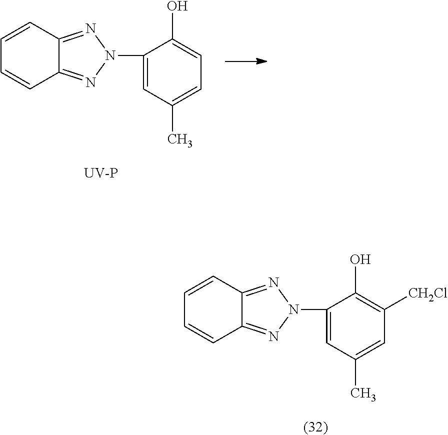

Add 350 g of 2-(2-hydroxy-5-methyl)benzotriazole (UV-P), 55 g of paraformaldehyde, 2000 g of acetic acid and 300 g of 35% hydrochloric acid in a 5000 ml reaction flask, and heat it to 60° C., then keep it warm. After reacting for 10 hours, samples were taken to monitor the reaction. After completion, it was cooled, washed with water, and dried to obtain 409 g of white powder (Compound 32) with a yield may over 90%, C14 H12 ClN3 O. Melting point: 163-164° C.

### Example 2, Preparation of 2-(2-hydroxy-3-(N,N-dimethylaniline)-5-methyl) benzotriazole (Compound 33)

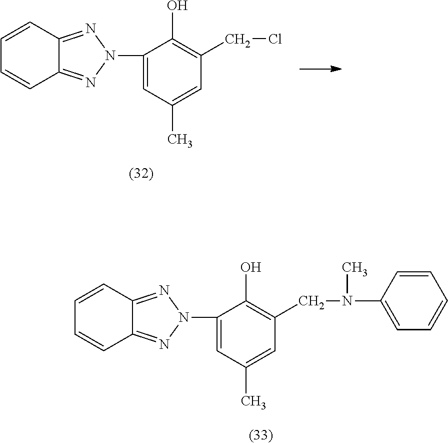

Put 180 ml of toluene, 17 g of potassium carbonate, 13.9 g of N-methylaniline, 0.2 g of phase catalyst, and 33.3 g of compound (32) in a reaction flask. The temperature was raised to 90-100° C., reacted for 5 hours, and samples were taken to monitor the reaction. Cool down to 30° C., wash with water and recover the toluene. Add 180 g of methanol and stir it, filter and dry to obtain 42 g of solid (compound 33). The yield is about 80%, C21H20N4O. Melting point: 98-100° C.

### Example 3, Preparation of 2-(2-hydroxy-3-(4-methanyl-N,N-dimethylaminophenyl)-5-methyl)benzotriazole (Compound 34)

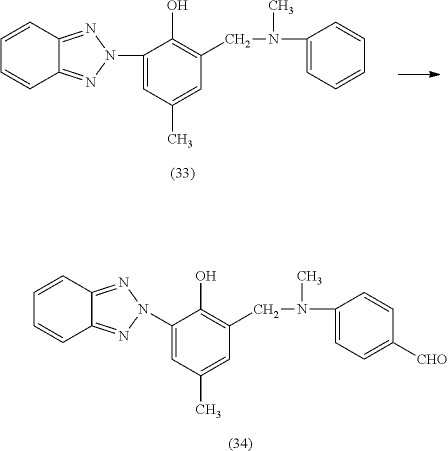

35 g of compound (33) and 8.7 g of DMF were added in a reaction flask, and 18.4 g of phosphorus oxychloride was added dropwise at room temperature. The temperature was raised to 90° C. for 2 hours, and samples were taken to monitor the reaction. The reaction material was slowly added to 300 g of water below 30° C. for hydrolysis. After the addition, the pH is adjusted to 8 with 30% caustic alkali liquids, and the solid was filtered out. Dissolved with 150 g of toluene, washed twice with water at 50° C., and cooled to 15° C. to precipitate 31.8 g of solid (Compound 34), with a yield of about 80%, C21H19N4O2. Melting point: 111˜113° C.

### Example 4, Preparation of (4-((3-(2H-benzo[d][1,2,3]triazol-2-yl)-2-hydroxy-5-methylbenzyl) (methyl) amino) benzylidene) dimethyl malonate (Compound 4, L-486)

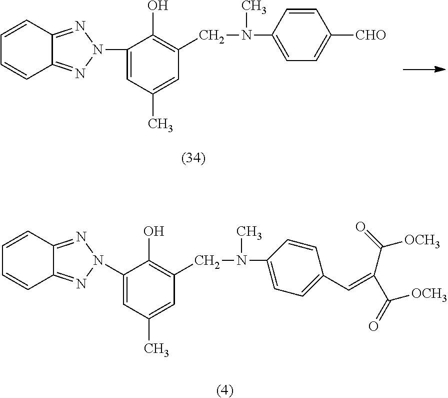 [SMILES: Cc1cc(CN(C)c2ccc(C=O)cc2)c(O)c(-n2nc3ccccc3n2)c1 ; "(34)" ; COC(=O)C(=Cc1ccc(N(C)Cc2cc(C)cc(-n3nc4ccccc4n3)c2O)cc1)C(=O)OC ; "(4)"]

Add 60 g of 2-(2-hydroxy-3-(4-methanyl-N,N-dimethyl-aminobenzene)-5-methyl)benzotriazole (compound 34), 21 g of dimethyl malonate ester, and 150 g of toluene in a reaction flask, and heating up. Then add 10 g of ammonium acetate and 20 g of acetic acid. First, react at 95° C. for 2 hours, then reflux to dehydrate (about 112° C.) for 6 hours. Monitor the reaction, and cool to 30° C. The solid was filtered, washed twice with water, and dried to obtain a yellow solid (compound 4), C₂₇H₂₆N₄O₅. Melting point: 191° C.-193° C.

### Example 5, Preparation of (4-((3-(2H-benzo[d][1,2,3] triazol-2-yl)-2-hydroxy-5-methylbenzyl)(ethyl)amino) benzylidene) dimethyl malonate (Compound 5, L-500)

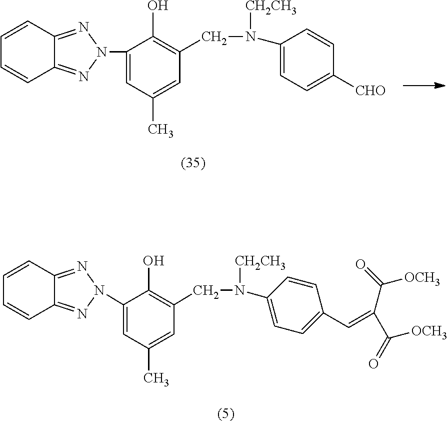 [SMILES: CCN(Cc1cc(C)cc(-n2nc3ccccc3n2)c1O)c1ccc(C=O)cc1 ; "(35)" ; CCN(Cc1cc(C)cc(-n2nc3ccccc3n2)c1O)c1ccc(C=C(C(=O)OC)C(=O)OC)cc1 ; "(5)"]

According to the method of Example 1-4, but use N-ethylaniline to replace N-methylaniline, to obtain 2-(2-hydroxy-3-(4-methanyl-N-ethyl-anilino)-5-Methyl) benzotriazole (Compound 35), C₂₂H₂₂N₄O₂. Melting point: 156-157° C. According to the method of Examples 1-4, dimethyl malonate was added to react to obtain compound (5), C₂₈H₂₈N₄O₅. Melting point: 173° C.-178° C.

### Example 6, Preparation of 3-(4-((3-(2H-benzo[d][1,2,3]triazol-2-yl)-2-hydroxy-5-methylbenzyl) (methyl) amino) phenyl) Ethyl-2-cyanoacrylate (Compound 6, L-467)

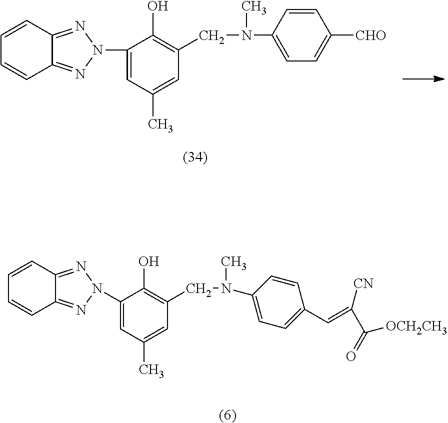 [SMILES: Cc1cc(CN(C)c2ccc(C=O)cc2)c(O)c(-n2nc3ccccc3n2)c1 ; "(34)" ; CCOC(=O)/C(C#N)=C/c1ccc(N(C)Cc2cc(C)cc(-n3nc4ccccc4n3)c2O)cc1 ; "(6)"]

Add 60 g of 2-(2-hydroxy-3-(4-methanyl-N,N-dimethylamino-benzene)-5-methyl) benzotriazole (Compound 34), 2-cyanoacetic acid ethyl acetate 18 g, toluene 150 g, in a reaction flask. Add 10 g of ammonium acetate and 20 g of acetic acid. First react at 95° C. for 2 hours, then reflux to dehydrate for 6 hours, and take samples to monitor the reaction. Cool to 30° C., filter out the solid, and the solid was washed and dried to obtain a solid (compound 6), C₂₇H₂₅N₅O₃. Melting point: 177-179° C.

### Example 7, Preparation of 2-(4-((3-(2H-benzo[d][1,2,3]triazol-2-yl)-2-hydroxy-5-methylbenzyl) (methyl) amino) benzylidene) diisopropyl malonate (Compound 7, L-542)

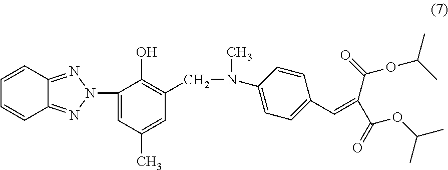 [SMILES: Cc1cc(CN(C)c2ccc(C=C(C(=O)OC(C)C)C(=O)OC(C)C)cc2)c(O)c(-n2nc3ccccc3n2)c1]

According to the method of Examples 1-4, but add diisopropyl malonate to replace methyl malonate. The reaction yields compound (7), C₃₁H₃₄N₄O₅. Melting point: 80° C.-87° C.

### Example 8, Preparation of 2-(4-((3-(2H-benzo[d][1,2,3]triazol-2-yl)-2-hydroxy-5-ethylbenzyl) (ethyl group) amino) benzylidene) diisopropyl malonate (Compound 8, L-556)

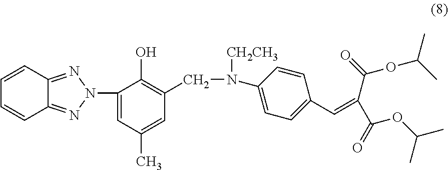 [SMILES: CCN(Cc1cc(C)cc(-n2nc3ccccc3n2)c1O)c1ccc(C=C(C(=O)OC(C)C)C(=O)OC(C)C)cc1]

According to the method of Example 7, but add N-ethylaniline to replace N-methylaniline, Compound (8, L-556), C₃₂H₃₆N₄O₅ was obtained. Melting point: 87° C.-90° C.

### Example 9, Preparation of 2-(4-((3-(2H-benzo[d][1,2,3] triazol-2-yl)-2-hydroxy-5-methylbenzyl) (methyl)amino) benzylidene) dipropionic acid-1-en-2-yl ester (Compound 9, L-538)

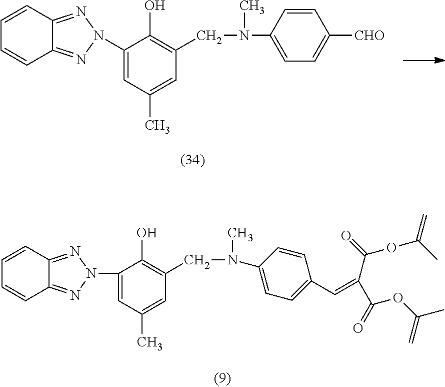 [SMILES: Cc1cc(CN(C)c2ccc(C=O)cc2)c(O)c(-n2nc3ccccc3n2)c1 ; C=C(C)OC(=O)C(=Cc1ccc(N(C)Cc2cc(C)cc(-n3nc4ccccc4n3)c2O)cc1)C(=O)OC(=C)C]

According to the method of Example 7, but add diisopropyl malonate instead of diisopropyl malonate. The compound (9), C₃₁H₃₀N₄O₅, was obtained. Melting point: 132-141° C.

### Example 10, Preparation of 2-(4-((3-(2H-benzo[d][1,2,3]triazol-2-yl)-2-hydroxy-5-methylbenzyl) (methyl)amino)benzylidene)di(bis(2-ethylhexyl)malonate (Compound 10, L-682)

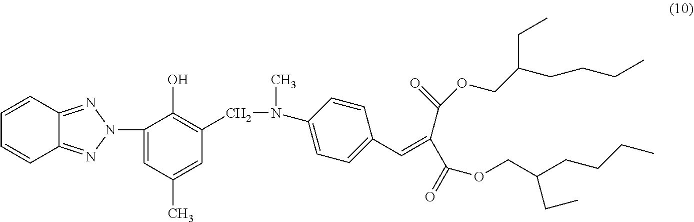 [SMILES: CCCCC(CC)COC(=O)C(=Cc1ccc(N(C)Cc2cc(C)cc(-n3nc4ccccc4n3)c2O)cc1)C(=O)OCC(CC)CCCC]

24 g of compound (34) was dissolved in toluene and heated to reflux at 110° C. in a condensing trap. To the toluene solution, 13 g 2-ethylhexanol and 1.5 g p-toluenesulfonic acid were added. Samples were taken to monitor the reaction. After the reaction completion, the product was purified by column chromatography to obtain compound (10), C₆₁H₉₄N₄O₅.

### Example 11, Preparation of 2-(4-((3-(2H-benzo[d][1,2,3] triazol-2-yl)-2-hydroxy-5-methylbenzyl) (methyl) amino) benzylidene) bis(octadecyl)malonate (Compound 11)

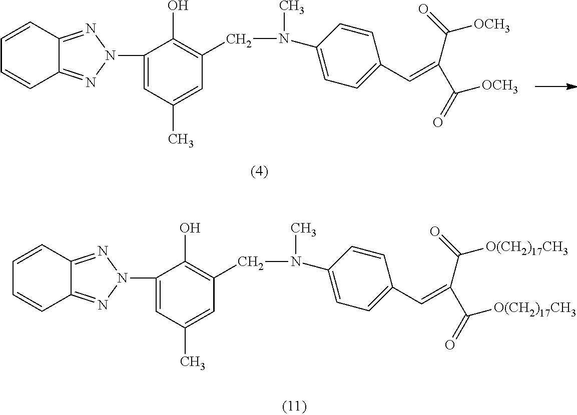

According to Example 10, but add stearyl alcohol instead of 2-ethylhexanol. Samples were taken to monitor the reaction. After the reaction completion, the product was purified by column chromatography to obtain compound (11), C₆₁H₉₄N₄O₅.

### Example 12, Preparation of 2-(4-((3-(2H-benzo[d][1,2,3] triazol-2-yl)-2-hydroxy-5-methylbenzyl) (methyl) amino) benzylidene) malonate polyether ester (Compound 12)

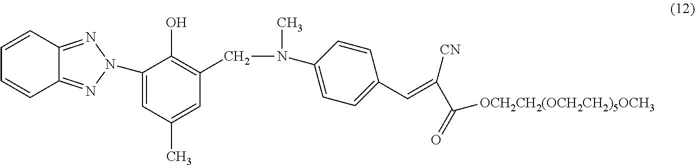

According to the method of Example 10, but add methoxypolyethylene glycol to replace 2-ethylhexanol. The reaction was monitored by HPLC. After the reaction completion, the product was purified by GPC (colloid permeation chromatography) to obtain the compound of formula (12).

### Example 13, Preparation of 2-(4-((3-(2H-benzo[d][1,2,3]triazol-2-yl)-2-hydroxy-5-(2,4,4-Trimethylpentan-2-yl)benzyl)(ethyl)amino)benzylidene)dimethylmalonate (Compound 13)

According to the method of Examples 1-4, 2-(2-hydroxy-5-tert-octylphenyl)-2H-benzotriazole (Tinuvin 329) was used instead of UV-P as the starting material. The compound (13), C₃₄H₃₈N₆O₄, was obtained.

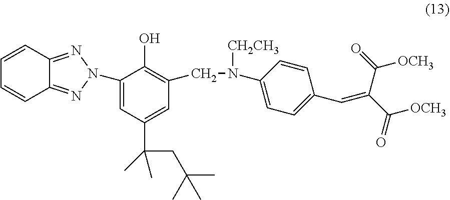 [SMILES: CCN(Cc1cc(C(C)(C)CC(C)(C)C)cc(-n2nc3ccccc3n2)c1O)c1ccc(C=C(C(=O)OC)C(=O)OC)cc1]

Tinuvin 329 was obtained from Eutec co. (Eusorb UV 329), or as described in preparation method 4. The preparation: 13.8 g of o-nitroaniline was added to 25 ml of 37% hydrochloric acid and stirred, diluted with 40 ml of water and cooled to −15° C. Add 7.5 g of sodium nitrite (dissolved in water) and keep the temperature at 0˜5° C. to obtain diazonium salt (Diazonium). Mix 5.2 g of 4-tert-Octylphenol, 20 ml of petroleum ether, 5 ml of water and 2.5 g of calcium hydroxide, and take samples to monitor the reaction. Add 20 g of ice to 0° C. Add the aforementioned diazonium salt and stir for 2 hours. Add concentrated hydrochloric acid to neutralize and dry to obtain 2-((2-nitrophenyl)diazenyl)-4-(2,4,4-trimethylpent-2-yl)phenol (compound 36), C₂₀H₂₅N₃O₃. Melting point: 114-115° C.

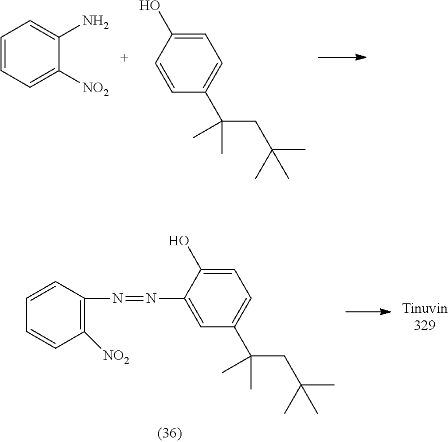 [SMILES: Nc1ccccc1[N+](=O)[O-] ; CC(C)(C)CC(C)(C)c1ccc(O)cc1 ; CC(C)(C)CC(C)(C)c1ccc(O)c(N=Nc2ccccc2[N+](=O)[O-])c1]

Take 35.7 g of compound (36) and dissolve in 100 ml of petroleum ether, add 17.2 g of zinc and 100 ml of water. At 50° C., 41.6 g of NaOH solution (25%) was added within 4 hours and left for 1 hour. 100 ml of concentrated hydrochloric acid was added and left for 2 hours, Samples were taken to monitor the reaction. The organic layer was washed with water, and the solvent was removed to obtain Tinuvin 329 compound. C₂₀H₂₅N₃O, Melting point: 102-106° C.

### Example 14, Preparation of 2-(4-((3-(5-chloro-2H-benzo[d][1,2,3]triazol-2-yl)-2-hydroxybenzyl) (ethyl) amino) benzylidene) dimethyl malonate (Compound 14)

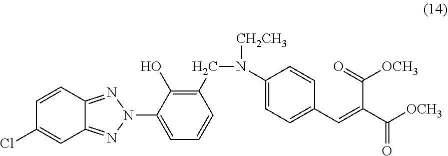 [SMILES: CCN(Cc1cccc(-n2nc3ccc(Cl)cc3n2)c1O)c1ccc(C=C(C(=O)OC)C(=O)OC)cc1]

According to the method of Example 13, but use 4-chloro-2-nitroaniline, instead of o-nitroaniline, as the starting material to obtain 2-(2′-hydroxy-phenyl)-5-chloro-benzotriazole (compound 37), C12H8ClN3O. Melting point: 139-140° C. According to the method of Example 1-4, but use 2-(2′-hydroxy-phenyl)-5-chloro-benzotriazole (compound 37) instead of (UV-P) as the starting material, compound (14) was obtained, C₂₇H₂₅ClN₄O₅.

### Example 15, Preparation of 2-(4-(ethyl(2-hydroxy-3-(5-methoxy-2H-benzo[d] [1,2,3]triazol-2-yl)benzyl) Amino) benzylidene) dimethyl malonate (Compound 15)

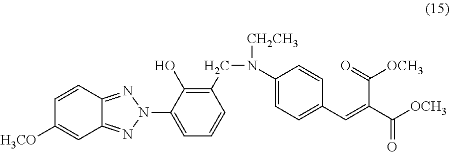 [SMILES: CCN(Cc1cccc(-n2nc3ccc(OC)cc3n2)c1O)c1ccc(C=C(C(=O)OC)C(=O)OC)cc1]

According to the method of Example 14, but use 4-methoxy-2-nitroaniline instead of 4-chloro-2-nitroaniline as the starting material to obtain 2-(2-hydroxy-phenyl)-5-methoxy-benzotriazole (Compound 38), C₁₄H₁₃N₃O₂. Melting point: 126-127° C.

According to the method of Example 1-4, but use 2-(2-hydroxy-phenyl)-5-methoxy-benzotriazole (compound 38) instead of (UV-P) as the starting material to obtain compound (15), C₂₉H₃₀N₄O₆.

### Example 16, Preparation of 3-(2H-benzo[d][1,2,3] triazol-2-yl)-5-((ethyl(4-((2,4,6-trioxo tetrahydro-pyrimidine-5(2H)-ylidene)methyl)phenyl)amino)methyl)-4-hydroxybenzoic acid methyl ester (Compound 16)

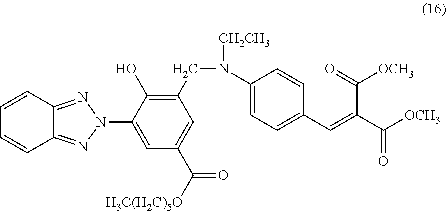

According to the method of Example 13, but replace 4-p-tert-octylphenol with 4-hydroxybenzoic acid, 3-(2H-benzo[d][1,2,3]triazol-2-yl)-4-Hydroxybenzoic acid. Then add sulphurous chloride, heat to reflux for 2 hours, evaporate the sulphurous chloride, add n-hexanol and reflux for 1 hour. Take samples to monitor the reaction. 3-(2H-benzo[d][1,2,3]triazol-2-yl)-4-hydroxybenzoic acid hexyl ester was obtained (Compound 39), C₁₉H₂₁N₃O₃. Melting point: 83-84° C. According to the method of Examples 1-4, but use compound (39) instead of UV-P as the starting material, and compound (16), C₃₄H₃₈N₄O₇ was obtained.

### Example 17, Preparation of 2-((1-(3-(2H-benzo[d][1,2,3]triazol-2-yl)-2-hydroxy-5-methylbenzyl)-3a, 7α Dihydro-1H-indol-3-yl) methylene) dimethyl malonate (Compound 17)

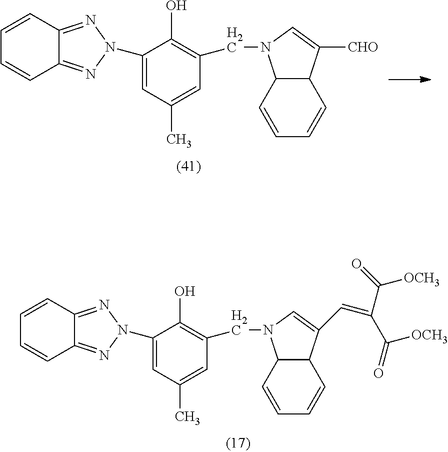 [SMILES: Cc1cc(CN2C=C(C=O)C3C=CC=CC32)c(O)c(-n2nc3ccccc3n2)c1 ; COC(=O)C(=CC1=CN(Cc2cc(C)cc(-n3nc4ccccc4n3)c2O)C2C=CC=CC12)C(=O)OC]

According to the method of Examples 1-4, 3-indolecarboxaldehyde (Compound 40) was used instead of N-methylaniline. Compound (41) was obtained. According to the method of Example 4, compound (41) was used instead of compound (34) and separated by column chromatography to obtain compound (17), C₂₈H₂₆N₄O₅, m/z: 498.2[M]⁺.

3-indole formaldehyde is can be bought or prepared in the following way (Vilsmeier reaction): In an ice bath, drop 16 g POCl3 into 30 g DMF within 30 minutes. 11 g indole compound was slowly added to the DMF solution and the temperature was raised to 35° C. The reaction was stirred for 1 hour. Add 50 g of crushed ice and stir to the obtained paste, and add 0.1M NaOH slowly while stirring. After washing with water, it was recrystallized with ethanol to obtain compound (40), C₉H₇NO. Melting point: 196˜197° C.

### Example 18, Preparation of 3-(4-((3-(2H-benzo[d][1,2,3]triazol-2-yl)-2-hydroxy-5-methylbenzyl) (methyl (amino) phenyl)-2-cyano-3-phenylethyl acrylate (Compound 18)

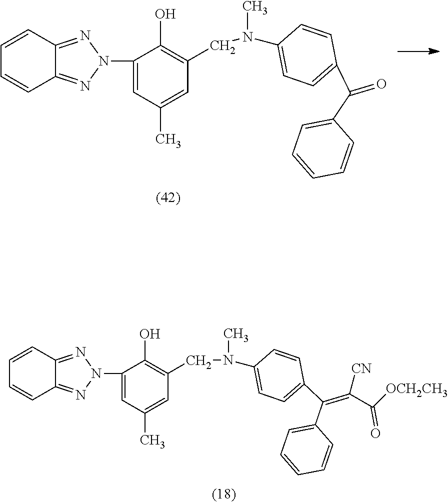 [SMILES: Cc1cc(CN(C)c2ccc(C(=O)c3ccccc3)cc2)c(O)c(-n2nc3ccccc3n2)c1 ; CCOC(=O)/C(C#N)=C(\c1ccccc1)c1ccc(N(C)Cc2cc(C)cc(-n3nc4ccccc4n3)c2O)cc1]

According to the method of Example 2, but use 4-methylaminobenzophenone instead of N-methylaniline, and compound (42) was obtained. According to the method of Example 4, but replace the compound (34) with the compound (42), and the mixture was separated by column chromatography to obtain compound (18), C₃₃H₂₉N₅O₃. m/z: 543.2 [M]⁺.

### Example 19, Preparation of 2,2′-((2-((3-(2H-benzo[d][1,2,3]triazol-2-yl)-2-hydroxy-5-methylbenzyl (ethyl) amino)-1,4-phenylene)bis(methanelidene))tetraethyl dimalonic acid (Compound 19)

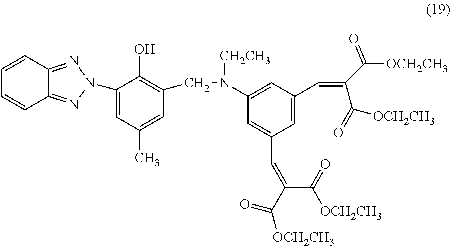 [SMILES: CCOC(=O)C(=Cc1cc(C=C(C(=O)OCC)C(=O)OCC)cc(N(CC)Cc2cc(C)cc(-n3nc4ccccc4n3)c2O)c1)C(=O)OCC]

According to the method of Example 2, but use dimethyl 5-(ethylamino)isophthalate (compound 43, melting point 118° C.) instead of N-methylaniline. The product was separated by column chromatography to obtain ((3-(2H-Benzo[d][1,2,3]triazol-2-yl)-2-hydroxy-5-methylbenzyl) (ethyl)amino)dimethyl isophthalate (Compound 44).

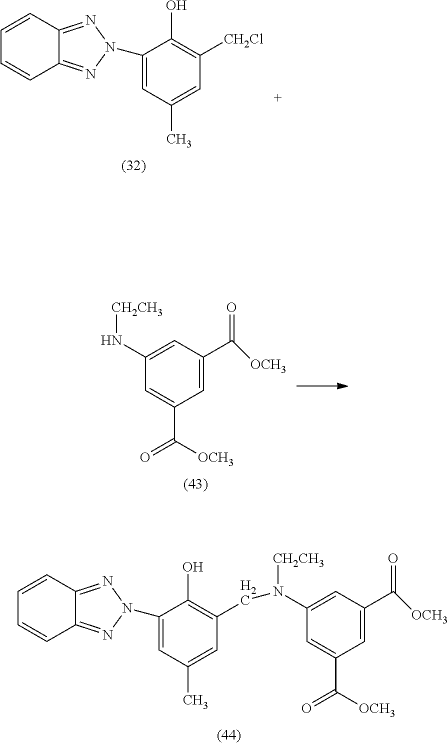

24 g of compound (44) was dissolved in 100 ml of toluene. At −78° C., under an argon atmosphere, 120 ml of a 1M toluene solution of diisobutylaluminum hydride (DIBAL-H) was added dropwise to the compound (44) solution. After adding DIBAH, continue stirring for 2 hours. Add methanol, then keep it at room temperature. Add 1M HCl, stir for 5 minutes, and extract it with ethyl acetate. After washing with saturated NaCl aqueous solution and drying with MgSO4, filtrate it and remove the solvent under reduced pressure, Compound (45) was obtained. According to the method of Example 4, but replace the compound (34) with compound (45). The product was separated by column chromatography to obtain the compound (19), C₃₈H₄₂N₄O₉. m/z: 698.3 [M]⁺.

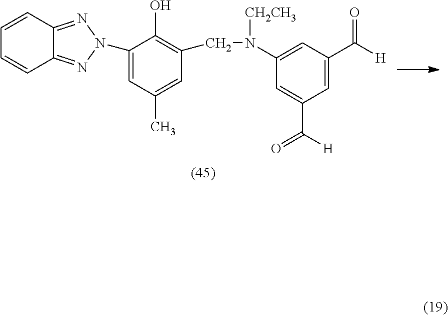 [SMILES: CCN(Cc1cc(C)cc(-n2nc3ccccc3n2)c1O)c1cc(C=O)cc(C=O)c1]

### Example 20, Preparation of 3-(4-(3-(3-(4,6-bis(2,4-dimethylphenyl)-1,3,5-triazin-2-yl)-5-(tert-butyl)-4-hydroxyphenyl)-N-methyl-propanamido) phenyl)-2-cyanoacrylate (Compound 20)

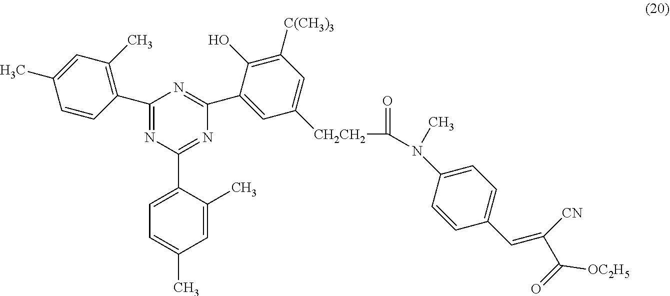

Take 5.2 g of 3-(3-(4,6-bis(2,4-dimethylphenyl)-1,3,5-triazin-2-yl)-5-(tert-butyl)-4-Hydroxyphenyl) methyl propionate (compound 46), dissolve in toluene, and heat to reflux at 110° C. in a flask equipped with a condensation trap. To the toluene solution, was added 3.7 g of ethyl 2-cyano-3-(4-(methylamino)phenyl) acrylate (Compound 47). Use HPLC to monitor the reaction. After the reaction was completed, vacuum it and separate with column chromatography. Compound (20) was obtained, C₄₅H₄₇N₅O₄, m/z: 721.4 [M]⁺.

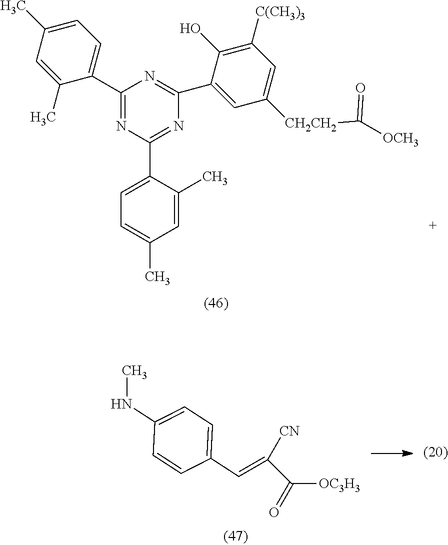

Preparation method of Compound (46), according to CN201710949552.0. Briefly, 16 g of 2-chloro-4,6-bis(2′,4′-phenyl)-1,3,5-triazine (Compound 48) and 15 g of methyl 3-(3-(tert-butyl)-4-hydroxyphenyl)propionate (Compound 49) were dissolved in 150 mL of chlorobenzene, and 10 g of anhydrous aluminum trichloride was added. Heat and stir to dissolve it. The temperature was raised to 90° C., and the reaction was monitored by HPLC. After the completion of the reaction, it was distilled under reduced pressure and subjected to silica gel column chromatography to obtain compound (46). preparation method of 2-cyano-3-(4-(methylamino)phenyl) ethyl acrylate (compound 47): 20 g of dimethyl malonate and 13.6 g of 4-methylaminobenzene formaldehyde were dissolved in dichloromethane. Add molecular sieve to remove water and install calcium chloride pipe for waterproof. Add 1 ml piperidine and 0.6 ml acetic acid, and heat the reaction at reflux temperature for 2 hours. During the reaction, add fresh molecular sieve. After the completion of the reaction, remove solvent, wash and dry to obtain compound (47).

### Example 21, Preparation of 2-(4-((3-(4,6-bis(2,4-dimethyl-phenyl)-1,3,5-triazin-2-yl)-5-(methyl)-4-hydroxybenzyl) (methyl)amino)benzylidene)dimethylmalonate (Compound 21)

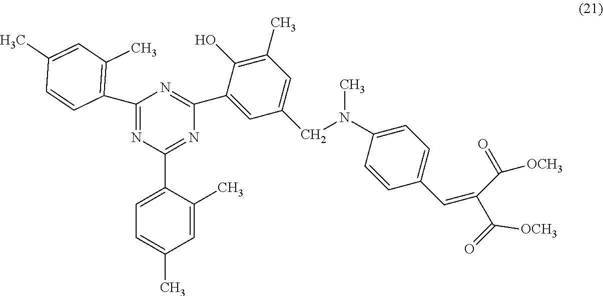 [SMILES: COC(=O)C(=Cc1ccc(N(C)Cc2cc(C)c(O)c(-c3nc(-c4ccc(C)cc4C)nc(-c4ccc(C)cc4C)n3)c2)cc1)C(=O)OC]

According to the method of Example 2, but use 2-(4,6-bis(2,4-dimethylphenyl)-1,3,5-triazin-2-yl)-6-(methyl)-4-(Chloromethyl)phenol (Compound 50) instead of Compound (32), and use dimethyl 2-(4-(methylamino)benzylidene) malonate (Compound 51) instead of N-methylaniline. After separation by column chromatography, compound (21) was obtained. C₄₀H₄₀N₄O₅, m/z: 656.3[M]⁺.

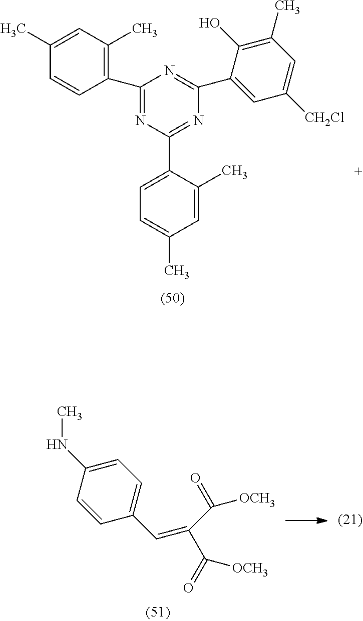

The preparation method of compound 50 is the same as in Example 20, but 4-(chloromethyl)-2-methylphenol is used instead of 3-(3-(tert-butyl)-4-hydroxyphenyl)propionate (49). The preparation method of dimethyl 2-(4-(methylamino) benzylidene)malonate (Compound 51) is the same as in Example 20, but dimethyl malonate is used instead of ethyl 2-cyanoacetate.

### Example 22, preparation of 2-(4-((2,4-di-tert-butyl-3-hydroxy-6-((2,4-di-tert-butylphenoxy)carbonyl)benzyl)(methyl)amino)benzylidene)malonate dimethyl and 2-(4-((3,5-di-tert-butyl-2-((3,5-di-tert-butyl-4-hydroxybenzyl) Mixture of ((methyl)amino)benzylidene) dimethyl malonate ((methyl)oxy)benzyl)(22)

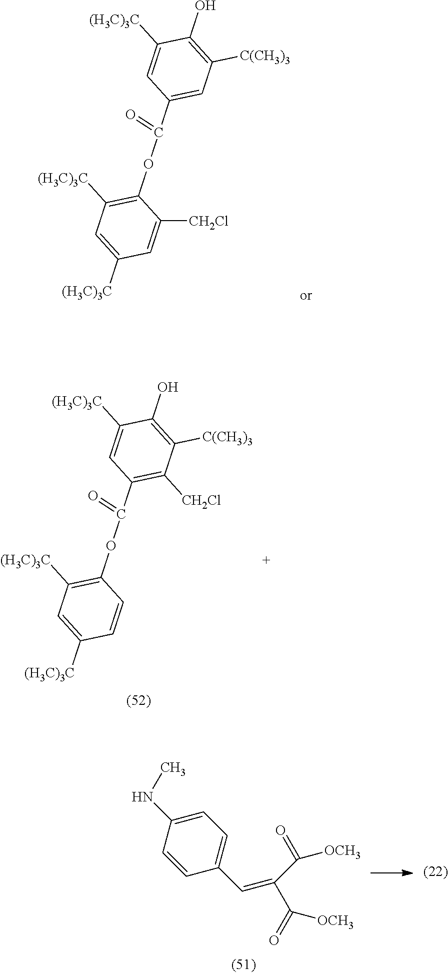

According to the method of Example 21, but use the mixture (52) of 3,5-di-tert-butyl-2-(chloromethyl)-4-hydroxybenzoic acid-2,4-di-tert-butylphenyl and 2,4-di-tert-butyl benzyl-6-(chloromethyl)phenyl 3,5-di-tert-butyl-4-hydroxybenzoate to replace 2-(4,6-bis(2,4-dimethyl) (Phenyl)-1,3,5-triazin-2-yl)-6-(methyl)-4-(chloromethyl)phenol (Compound 50) to obtain a mixture (22).

Preparation of mixture of 3,5-di-tert-butyl-2-(chloromethyl)-4-hydroxybenzoic acid-2,4-di-tert-butylphenyl ester and 2,4-di-tert-butyl-6-(chloromethyl)) phenyl 3,5-di-tert-butyl-4-hydroxy-benzoate: as in Example 2, but use 2,4-di-tert-butylphenyl-3,5-di-tert-butyl-4-Hydroxybenzoate (Eutec co., Eusorb UV-120) instead of UV-P.

### Example 23, preparation of (4-((5-benzyl-4-hydroxy-2-(octyloxy)benzyl)(methyl)amino)benzylidene) dimethyl malonate (Compound 23)

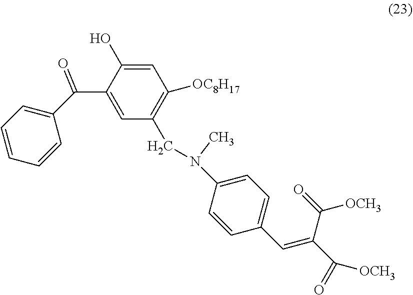

According to the method of Example 21, but use 5-(chloromethyl)-2-hydroxy-4-(octyloxy)phenyl)(phenyl)methanone (compound 53) instead of (compound 50) to obtain compound (23), C₃₅H₄₁NO₇, m/z: 587.3 [M]⁺.

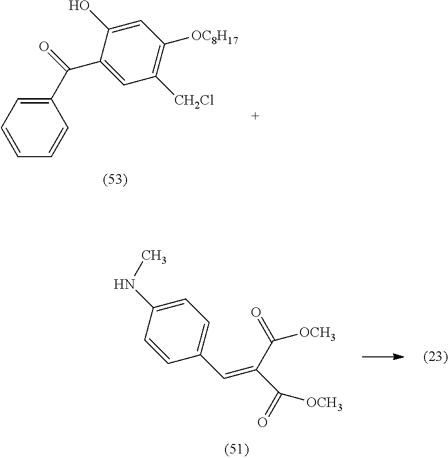

The preparation method of 5-(chloromethyl)-2-hydroxy-4-(octyloxy)phenyl)(phenyl)methanone (Compound 53): as in Example 2, but use 2,4-di-tert butylphenyl-3,5-di-tert-butyl-4-hydroxybenzoate (Eutec co., Eusorb UV-120) instead of UV-P.

### Example 24, Preparation of 2-(4-(4-(4-oxo-4H-benzo[d][1,3]oxazin-2-yl)benzyl)amino)benzylidene) dimethyl malonate (Compound 24)

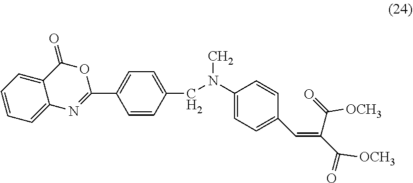 [SMILES: COC(=O)C(=Cc1ccc(N(C)Cc2ccc(-c3nc4ccccc4c(=O)o3)cc2)cc1)C(=O)OC]

According to the method of Example 21, but use 2-(4-(chloromethyl)phenyl)-4H-benzo[d][1,3]oxazin-4-one (compound 54) instead of 2-(4, 6-bis(2,4-dimethylphenyl)-1,3,5-triazin-2-yl)-6-(methyl)-4-(chloromethyl)phenol (compound 50), to obtain 2-(4-(4-(4-oxo-4H-benzo[d][1,3]oxazin-2-yl)benzyl)amino)benzylidene)dimethylmalonate (compound 24), C₂₈H₂₄N₂O₆, m/z: 484.2[M]⁺.

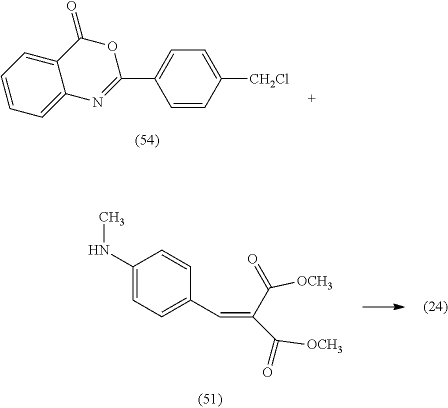

The preparation method of 2-(4-(chloromethyl)phenyl)-4H-benzo[d][1,3]oxazin-4-one (compound 54): 14 g of 2-aminobenzoic acid (compound 55) and 11g of triethylamine were added to 100 ml of dichloroethane, and 19 g of 4-chloromethylbenzyl chloride (Compound 56) was dropped and stirred to obtain Compound (54).

The preparation method of compound (56): 4-(hydroxymethyl)benzoic acid (57) was chlorinated in dichloromethane and refluxed with thionyl chloride to obtain compound (56), melting point: 28° C.

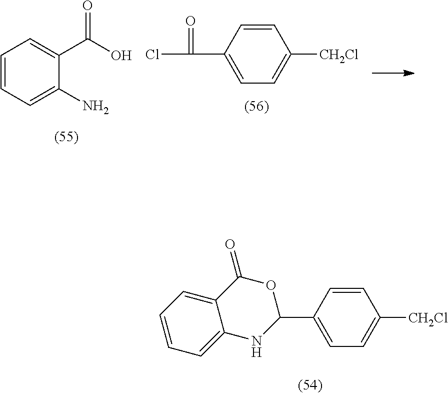

### Example 25, preparation of 2-(4-(Methyl((4-oxo-2-(4-(4-oxo-4H-benzo[d] [1,3]oxazin-2-yl)phenyl)-4H-Benzo[d][1,3] oxazine-6-yl)methyl)amino)benzylidene)dimethylmalonate (Compound 25)

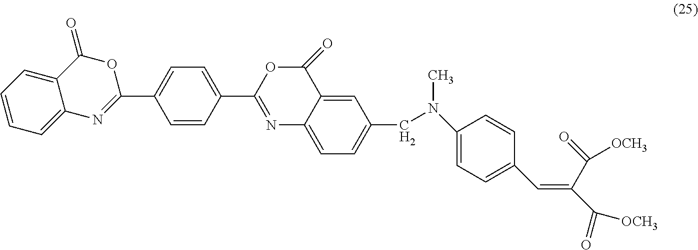 [SMILES: COC(=O)C(=Cc1ccc(N(C)Cc2ccc3nc(-c4ccc(-c5nc6ccccc6c(=O)o5)cc4)oc(=O)c3c2)cc1)C(=O)OC]

In 200 ml of toluene, add 40 g of 2-amino-5-(((4-(3-methoxy-2-(methoxycarbonyl)-3-oxoprop-1-en-1-yl) phenyl) (methyl) amino) methyl) benzoic acid (compound 59), 11 g of triethylamine, and 29 g of 4-(4-oxo-4H-benzo[d][1,3]oxazine-2-yl) benzoyl chloride (compound 58) with stirring. And the product was separated by column chromatography to obtain compound (25), C₃₆H₂₇N₃O₈, m/z: 629.2[M]⁺.

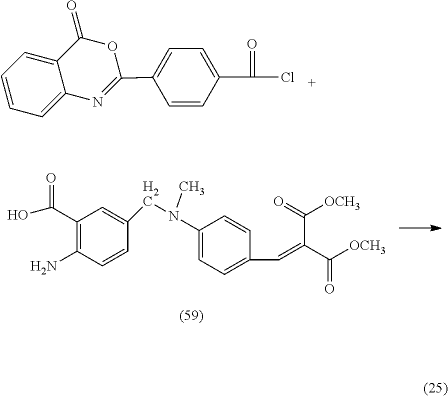 [SMILES: O=C(Cl)c1ccc(-c2nc3ccccc3c(=O)o2)cc1 ; COC(=O)C(=Cc1ccc(N(C)Cc2ccc(N)c(C(=O)O)c2)cc1)C(=O)OC]

The preparation method of compound (58): as in Example 24, but use methyl 4-(chlorocarbonyl)benzoate (60) instead of compound (56) to obtain compound (58).

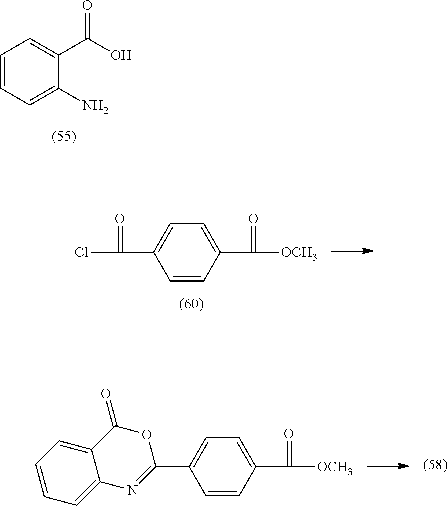 [SMILES: Nc1ccccc1C(=O)O ; COC(=O)c1ccc(C(=O)Cl)cc1 ; COC(=O)c1ccc(-c2nc3ccccc3c(=O)o2)cc1]

The preparation method of compound (59): as in Example 21, but use methyl 4-(chlorocarbonyl)benzoate (compound 61) instead of compound (54) to obtain compound (59).

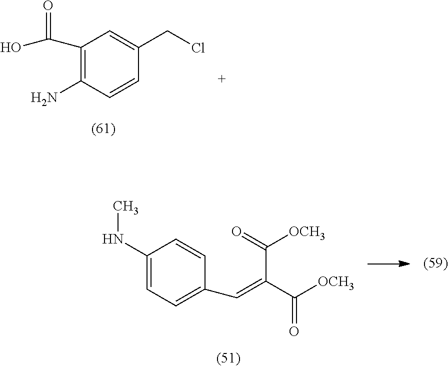 [SMILES: Nc1ccc(CCl)cc1C(=O)O ; CNc1ccc(C=C(C(=O)OC)C(=O)OC)cc1]

### Example 26, preparation of 2-(4-(((2,4-dihydroxy-quinolin-3-yl) methylene)amino)benzyl) (methyl)amino) benzylidene)propane dimethyl acid (Compound 26, L-3701)

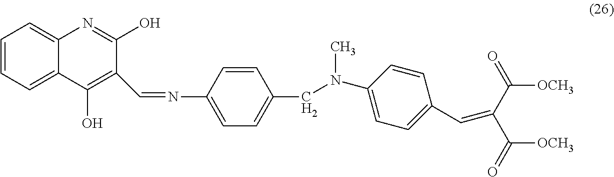 [SMILES: COC(=O)C(=Cc1ccc(N(C)Cc2ccc(N=Cc3c(O)nc4ccccc4c3O)cc2)cc1)C(=O)OC]

According to Example 21, use 3-(((4-(chloromethyl) phenyl)imino)methyl)quinoline-2,4-diol (compound 62) instead of compound (51) to obtain compound (26), C₃₀H₂₇N₃O₆, m/z: 525.2 [M]⁺.

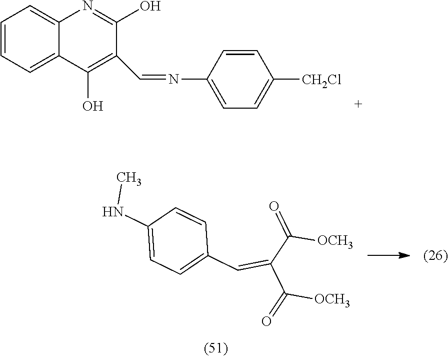

The preparation method of 3-(((4-(chloromethyl)phenyl) imino)methyl)quinoline-2,4-diol (62): as in Example 2, but use 3-((Phenylimino)methyl)quinoline-2,4-diol (UA-3701, melting point 194° C.) instead of UV-P.

### Example 27, Preparation of 2-(4-((4-(N′-(4-(ethoxy-carbonyl)phenyl)-N-methyliminomethamido)benzyl)(methyl)amino)benzylidene)dimethyl malonate (Compound 27, UV-1)

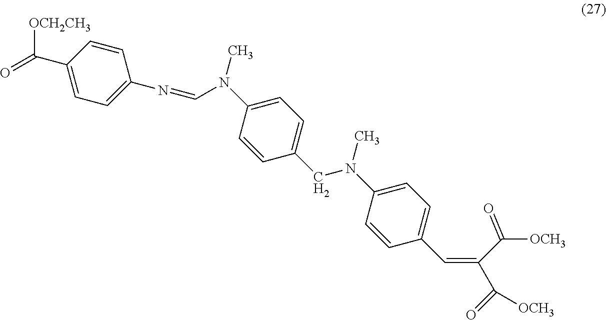 [SMILES: CCOC(=O)c1ccc(N=CN(C)c2ccc(CN(C)c3ccc(C=C(C(=O)OC)C(=O)OC)cc3)cc2)cc1]

According to Example 21, but use 4-((((4-(chloromethyl) phenyl)(methyl)amino)methylene)amino) ethyl benzoate (Compound 62) instead of compound (54), to obtain compound (27), C₃₁H₃₃N₃O₆, m/z: 543.24 [M]⁺.

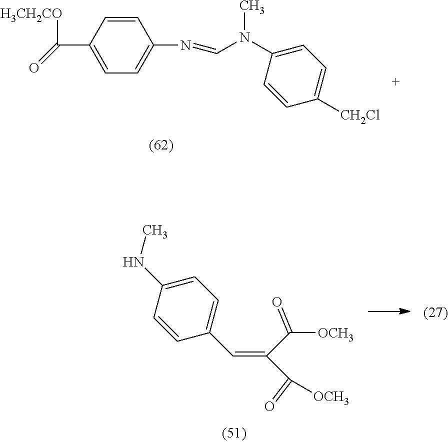

The preparation method of Compound (62): as in Example 2 method, but use 4-(((phenyl)(methyl)amino)methylene)amino) ethyl benzoate (Eutec co., UV-1, melting point 137° C.) instead of UV-P.

### Example 28, preparation of 2-(4-((3-ethoxy-4-(2-((2-ethylphenyl)amino)-2-oxoacetamido)benzyl)(methyl)amino)benzylidene)dimethyl malonate (Compound 28)

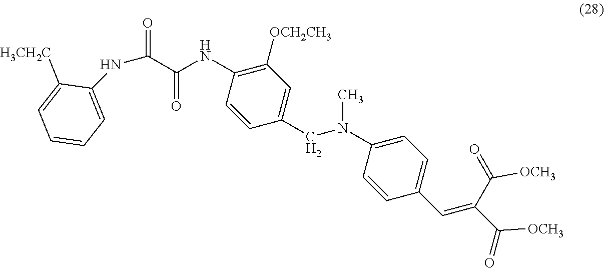

According to Example 21, but use 4-((((4-(chloromethyl) phenyl)(methyl)amino)methylene)amino)ethyl benzoate (compound 63) instead of compound (54), Compound (28), C₃₂H₃₅N₃O₇, was obtained. m/z: 573.3 [M]⁺.

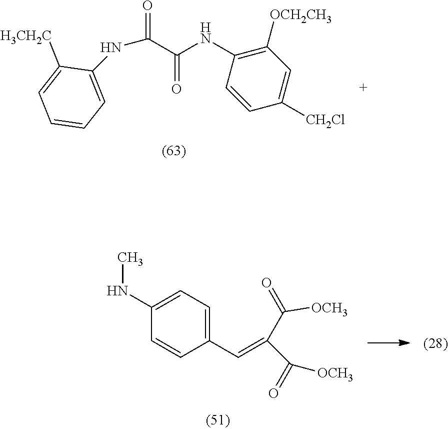

The preparation method of 4-((((4-(chloromethyl)phenyl)(methyl) amino) methylene) amino) ethyl benzoate (compound 63): as in Example 2, but replace UV-P with 4-(((methyl(phenyl) amino)methylene)amino)ethyl benzoate (Eutec co., UV-312, melting point 120° C.).

### Example 29, Preparation of 2-(4-(((9H-carbazole-3-yl)methyl)(methyl)amino)benzylidene)malonate (Compound 29)

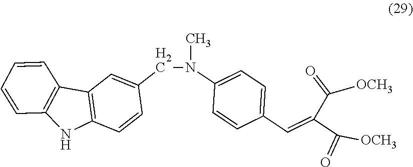 [SMILES: COC(=O)C(=Cc1ccc(N(C)Cc2ccc3[nH]c4ccccc4c3c2)cc1)C(=O)OC]

According to Example 21, but use 3-(chloromethyl)-9H-carbazole (Compound 64), instead of Compound (54). Compound (29), C₂₆H₂₄N₂O₄, was obtained. m/z: 428.2 [M]⁺.

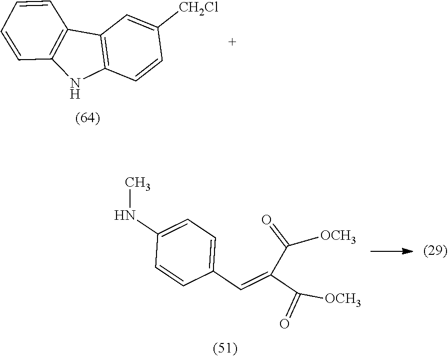

The preparation method of 3-(chloromethyl)-9H-carbazole (compound 64): as in Example 2, but use 9H-carbazole instead of UV-P.

### Example 30, Preparation of 2-cyano-3-(4-(N-methyl-9H-carbazole-1-carboxamido)phenyl) ethyl acrylate (Compound 30)

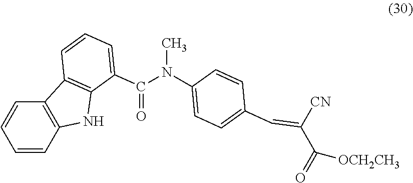 [SMILES: CCOC(=O)/C(C#N)=C/c1ccc(N(C)C(=O)c2cccc3c2[nH]c2ccccc23)cc1]

According to the method of Example 20, but use 9H-carbazole-1-carboxylic acid methyl ester (Compound 65) instead of Compound (54). Compound (30), C₂₆H₂₁N₃O₃, was obtained. m/z: 423.2[M]⁺.

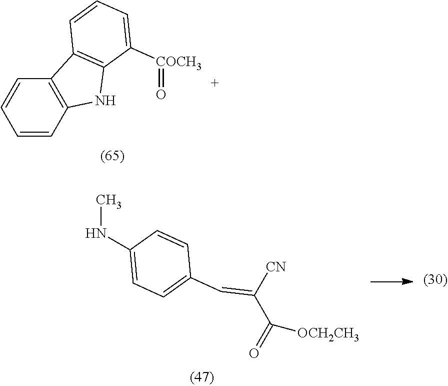

The preparation method of 9H-carbazole-1-carboxylic acid methyl ester: Using concentrated sulfuric acid as a catalyst, 9H-carbazole-1-carboxylic acid was in excess methanol with reflux to obtain compound (65).

### Example 31, Preparation of dimethyl 2-(4-((dibenzo[b,d] thiophen-2-yl-methyl)(methyl)amino)benzylidene) malonate (Compound 31)

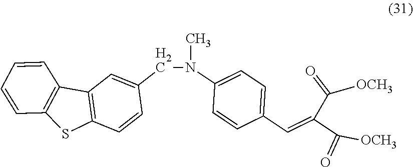 [SMILES: COC(=O)C(=Cc1ccc(N(C)Cc2ccc3sc4ccccc4c3c2)cc1)C(=O)OC]

According to the method of Example 21, but use 2-(chloromethyl)dibenzo[b,d]thiophene (compound 66) instead of compound (54). Compound (31) was obtained. C₂₆H₂₃NO₄S, m/z: 445.1 [M]⁺.

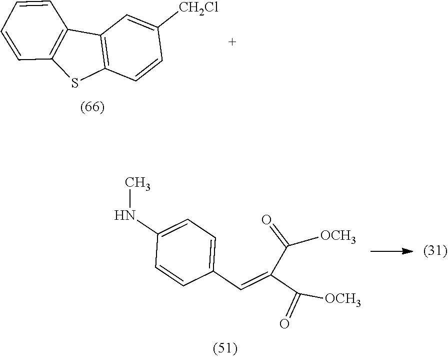

The preparation method of 2-(Chloromethyl)dibenzo[b,d]thiophene: as in Example 2, but use dibenzo[b,d]thiophene instead of UV-P.

### Example 32 Thermal Stability Analysis

UV or blue light blocking agents usually need to be processed at high temperatures or/and used outdoors. However, most ultraviolet or blue light blocking agents cannot maintain high stability at high temperatures. Here, we used the commercially available ultraviolet blocking compound, UV-P, or/and the blue light blocking compound, blue-1, as controls, and compared the thermal stability (thermogravimetric analyzer, TGA) with the current example compounds. The greater the weight loss, the worse the stability is.

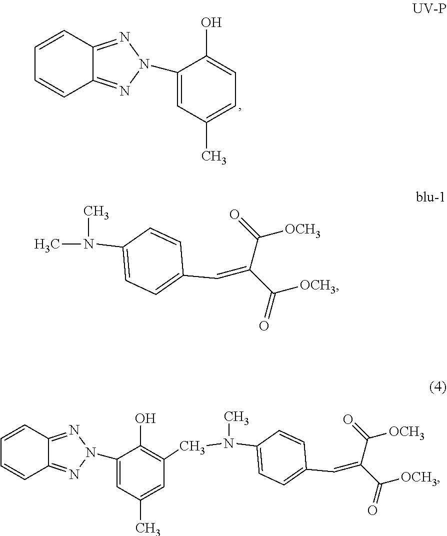 [SMILES: Cc1ccc(O)c(-n2nc3ccccc3n2)c1 ; COC(=O)C(=Cc1ccc(N(C)C)cc1)C(=O)OC ; COC(=O)C(=Cc1ccc(N(C)Cc2cc(C)cc(-n3nc4ccccc4n3)c2O)cc1)C(=O)OC]

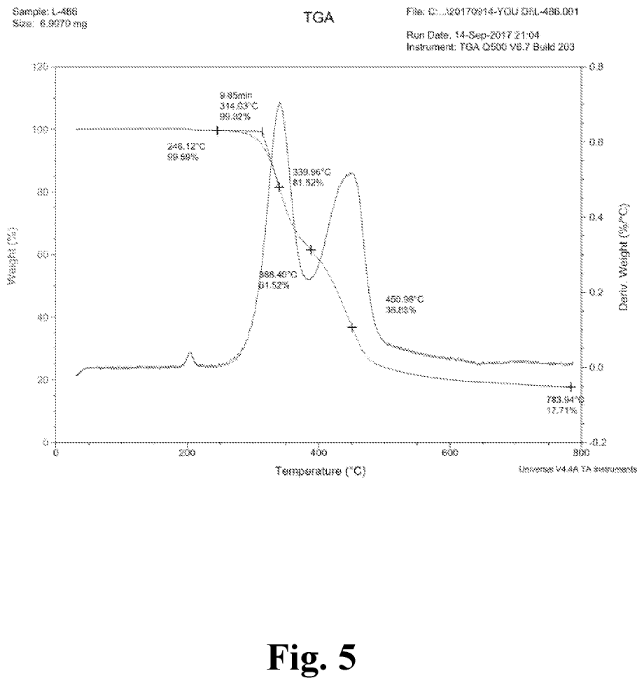

For UV-P, the weight loss caused by heat is 5% at 190° C. For blue-1, the weight loss caused by heat is 5% at 178.3° C. These agents cannot be used in high temperature processing of plastics. Surprisingly, the TGA of FIG. 5 shows that the compound of Example 4 (L-486) of the present invention has a thermal weight loss of less than 1% when the temperature is raised to 300° C. The TGA in FIG. 6 shows that when the temperature of the compound of Example 6 (L-467) reaches 300° C., the thermal weight loss is less than 1%. These compounds of the present invention can be used in polycarbonate (PC) processing. For the example compounds of the present invention, the temperature for 5% weight loss is above 200° C. They can be used for most plastic processing.

TABLE 1
Thermal stability

| | | | Compounds of |
|---|---|---|---|
| | Compound | Compound | ex. 4, ex. 6 |
| | UV-P | blue-1 | (FIG. 5, 6) |
| Decomposition | 190° C. | 178.3° C. | above 330° C. |
| temperature (5%) | | | |

### Example 33. Absorption Analysis for Blue Light

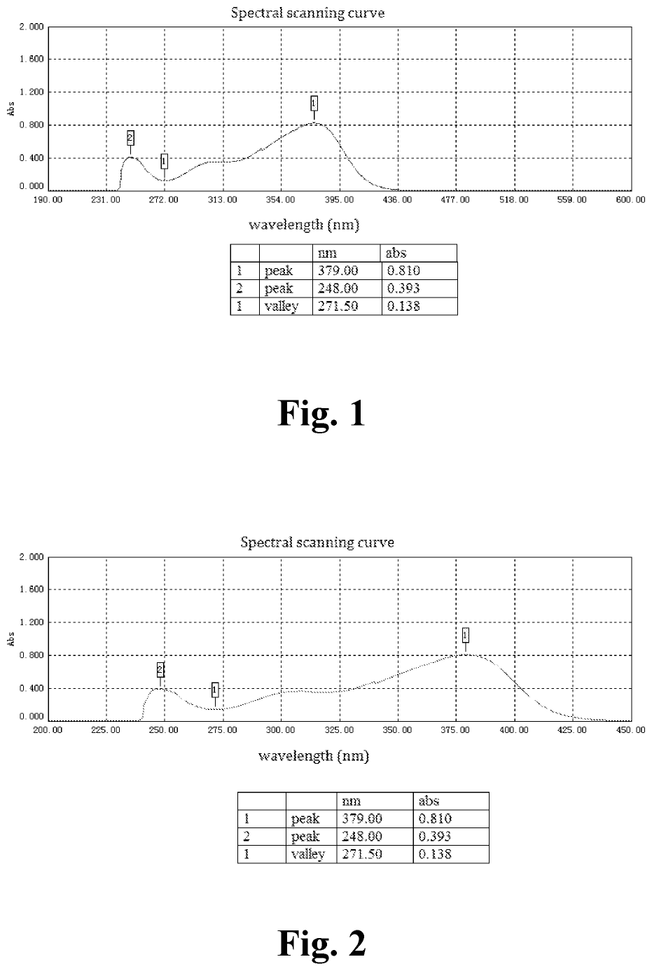

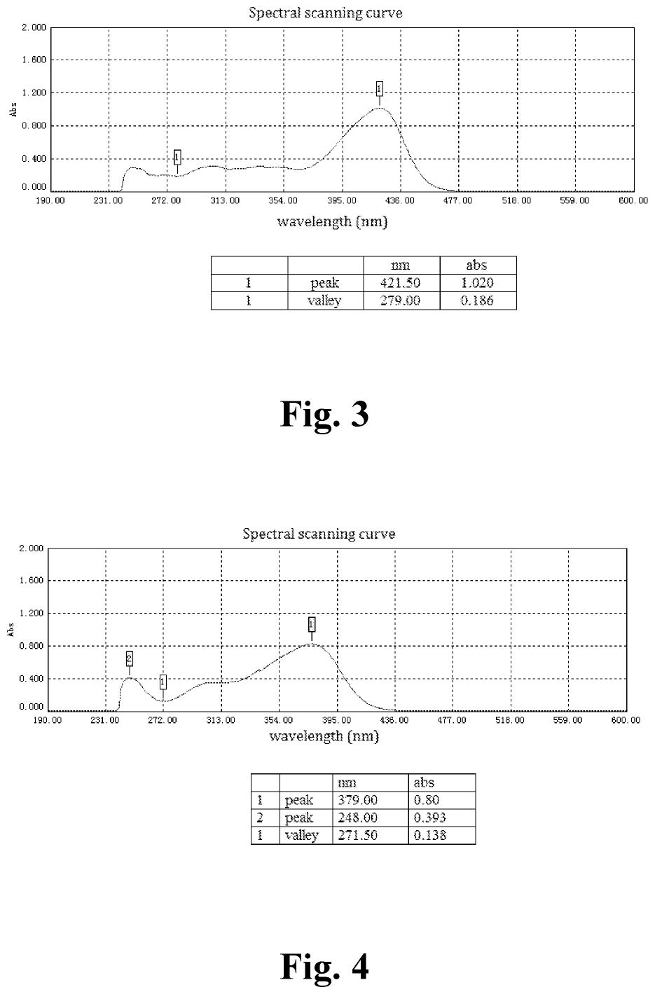

UVA (about 320-400 nm) can penetrate glass, is the main indoor ultraviolet light band. UVB (about 290-320 nm) is the main ultraviolet light band that causes photobiological effects of solar radiation on the skin. In many applications, simultaneous absorption of UVA, UVB and blue light is expected. UVA (about 320-400 nm). For example, FIGS. 1-4 are the UV-VIS absorption graphs of 10 mg/L in chloroform. It is the typical UV-VIS absorption pattern of the compound of the present invention. FIGS. 1-4 show that compounds of example simultaneously absorb ultraviolet light and blue light. And the absorption of long-wavelength blue light gradually decreases (indicating that the transmitted blue light has better color visual effects).

TABLE 2
Absorption analysis for blue light

| | | | Compounds of |
|---|---|---|---|
| | | Compounds | example |
| | Compound | of example | 4-5, 7-11, |
| | blue-1 | 6, 12, 18 | 13-17, 19-31 |
| | UVA + | UVA + UVB + | UVA + UVB + |
| | blue light | blue light | blue light |
| Evaluation of | + | +++ | +++ |
| absorption range | | | |
| Absorption (max.) | about 380 nm | about 420 nm | about 380 nm |
| Evaluation of color | +++ | +++ | +++ |
| visual effect | | | |
| Evaluation of | — | +++ | +++ |
| thermal stability | | | |

1. An anti-blue light compound of formula (1), 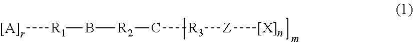
R₁˜R₃ are a bond or/and any divalent linking groups;
A˜C are unsubstituted or one or more R₄ substituted benzene ring, benzo carbocyclic ring, nitrogen-containing heterocyclic ring, or benzo nitrogen-containing heterocyclic ring;
R₄ is each independently selected from hydrogen, halogen, hydroxyl, amino, nitro, cyano, linear or branched C₁˜C₁₈ alkyl, C₁˜C₁₈ alkenyl, phenyl, OR₅, SR₅, SO₂R₅, SO₃R₅, COOR₅, COR₅, OCOR₅, C(O)NR₆R₇, SO₂NR₆R₇, and NR₆R₇, wherein R₅, R₆, and R₇ are independently hydrogen, or linear or branched C₁˜C₈;
X is one or more groups, each independently selected from COOR₈, CN, CONR₆R₇ and COR₈, wherein R₈ is selected from H, linear or branched C₁˜C₁₈ alkyl, C₁˜C₁₈ alkenyl, and polyethylene glycol with molecular weight 50˜1000 daltons;
Z is a carbon atom, Z and R₃ are connected via single, or double, or triple bond;
Z and X, are connected via 1˜3 single bonds, wherein n=1˜3;
[A]r ring and R₁ are connected via 1 or 2 single bonds, wherein r=1˜2;
C ring and R₃ are connected via 1 or 2 or 3 single bonds, wherein m=1˜3.

2. The anti-blue light compound according to claim 1, wherein,
R₁˜R₃ is a bond, or/and a chain consisting of 1-10 groups selected from the following:—O—, —S—, —C(═O)—, —COO—, —C(═S)—, —C(═NR₉)—, —CH₂—, —CH(R₉)—, —C(R₉)₂—, —C(R₉)═, —C≡, —C(R₉)═C(R₉)—, —N(R₉)—, —C(R₉)═N—, and phenyl;
R₉ is H, linear or branched C₁˜C₈ alkyl, unsubstituted phenyl, or phenyl substituted by OH, halogen, C₁˜C₄ alkoxy, linear or branched C₁˜C₄ alkyl;
Z and R₃ are connected via a double bond;
Z and X are connected via two single bonds, wherein n=2.

3. The anti-blue light compound according to claim 1, including at least one blue light absorbing group selected from: 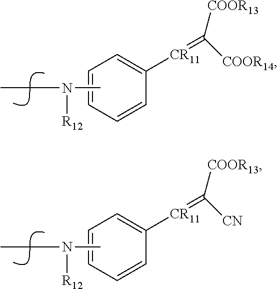
wherein, R₁₁˜R₁₄ are the same or different from each other independently selected from H, linear or branched C₁˜C₁₈ alkyl or alkenyl, and unsubstituted phenyl, or OH, halogen, C₁˜C₄ alkoxy, linear or branched C₁˜C₄ alkyl substituted phenyl,
and at least one ultraviolet light absorbing group selected from

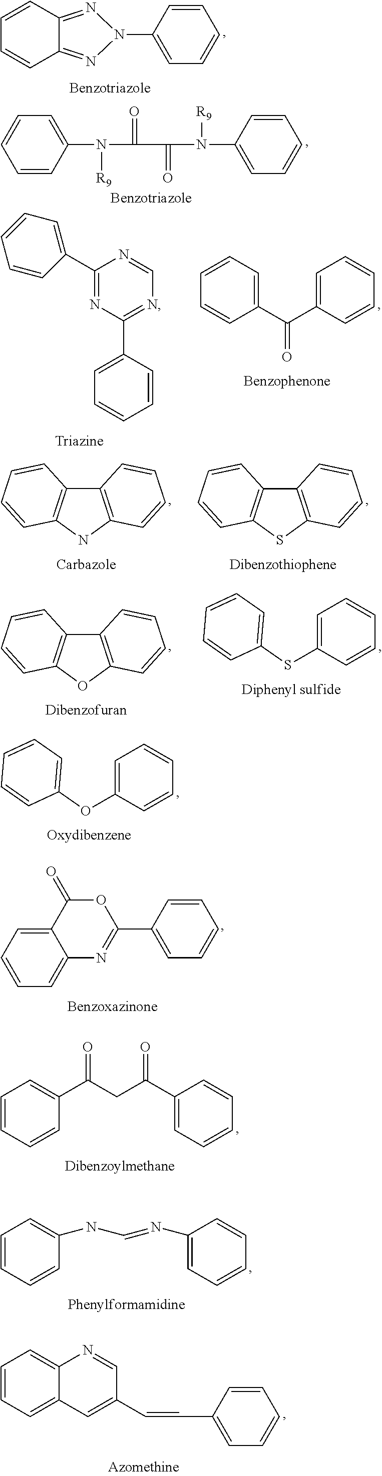

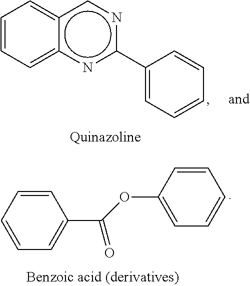

4. The anti-blue light compound according to claim 1, wherein,
A is selected from: 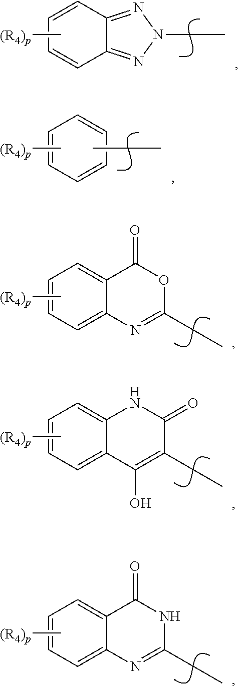
B is selected from: 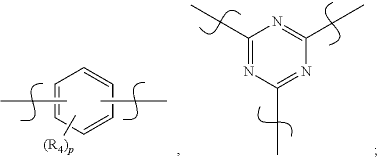
C is selected from: 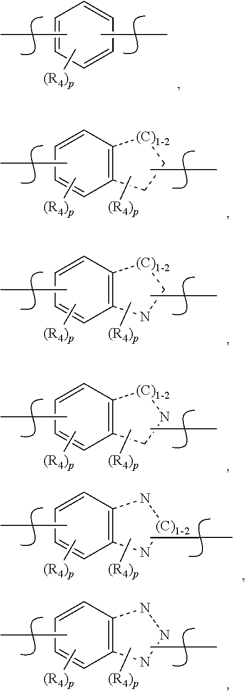
R₄ is one or more substituents, and each is independently selected from hydrogen, halogen, nitro, cyano, linear or branched C₁˜C₈ alkyl, C₁˜C₈ alkenyl, OR₅, SR₅, SO₂R₅, COOR₅, COR₅, C(O)NR₆R₇, and NR₆R₇, wherein R₅, R₆, and R₇ are each independently hydrogen, or a linear or branched C₁˜C₈ alkyl group,
p=1˜3.

5. The anti-blue light compound according to claim 4, wherein, 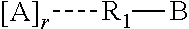
is selected from: 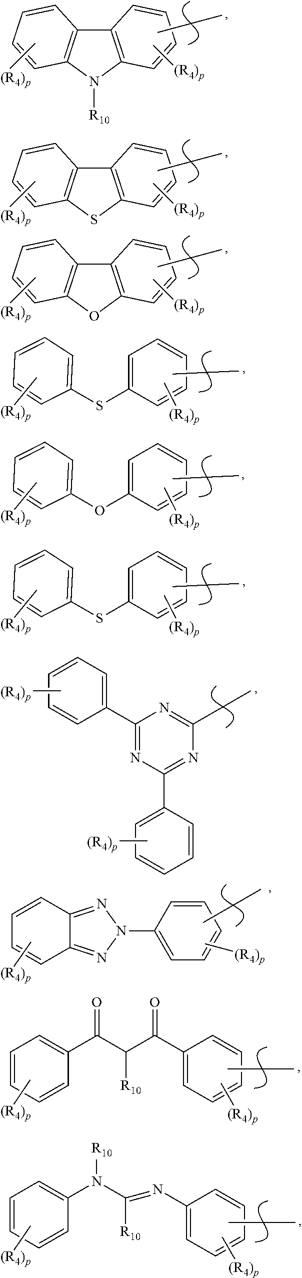 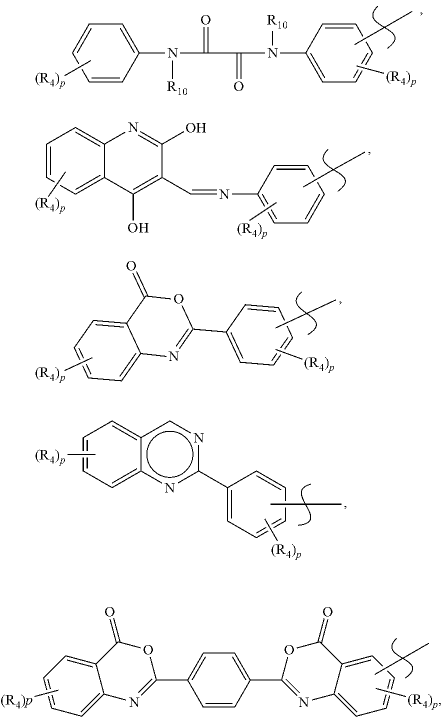
wherein, R₄ is one or more substituents, and each is independently selected from hydrogen, halogen, nitro, cyano, straight or branched C₁˜C₈ alkyl, C₁˜C₈ alkenyl, OR₅, SR₅, SO₂R₅, COOR₅, COR₅, C(O)NR₆R₇, and NR₆R₇, wherein, R₅, R₆, and R₇ are each independently hydrogen, or a linear or branched C₁˜C₆ alkyl group,
R₁₀ is H, linear or branched C₁˜C₈ alkyl, unsubstituted phenyl, or phenyl substituted by OH, halogen, C₁˜C₄ alkoxy, linear or branched C₁˜C₄ alkyl, p=1˜3.

6. The anti-blue light compound according to claim 1, wherein,
A is selected from: 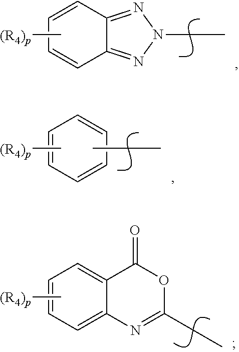
B is selected from: 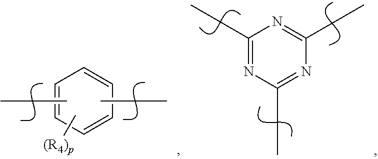
C is 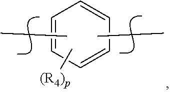
wherein, R₁ and R₃ are a bond, or/and a chain consisting of 1˜6 selected from the following groups:
—O—, —N(R₉)—, —C(═O)—, —COO—, —CH₂—, —CH(R₉)—, —C(R₉)₂—, —C(R₉)═, —C≡, —C(R₉)═N—, —NH—C(═O)—C(═O)—NH—, —(R₉)N—CH═N—, phenyl,
wherein, R₂ is a bond, —(CHR₉)_{q} N(C═O)(R₉)—, or —(CHR₉)_{q} N(R₉)—, R₄ is one or more substituents, and each is independently selected from hydrogen, halogen, linear or branched C₁˜C₈ alkyl, C₁˜C₈ alkenyl, OR₅, SR₅, COOR₅, COR₅, C(O)NR₆R₇, and NR₆R₇, wherein R₅, R₆, and R₇ are each independently hydrogen, or a linear or branched C₁˜C₆ alkyl group,
X is 1˜2 substituents, each independently selected from COOR₈, CN, CONR₆R₇ and COR₈, wherein, R₈ is selected from H, linear or branched C₁˜C₈ alkyl, and C₁˜C₈ alkenyl, R₉ is H, linear or branched C₁˜C₆ alkyl or phenyl, Z and R₃ are connected via a double bond,
m=1, n=1˜2, p=1˜3, q=0˜6.

7. The anti-blue light compound according to claim 6, wherein,
C is a divalent benzene ring;
R₂ is —CH₂ N(CH₂)— or —CH₂ N(CH₂CH₃)—;
n=2, and X is the same or different and each independently selected from COOR₈, and CN;
R₈ is selected from a linear or branched C₁˜C₈ alkyl group and a C₁˜C₈ alkenyl group.

8. The anti-blue light compound according to claim 7, wherein, A is selected from benzotriazole and benzene ring, B is a divalent benzene ring, when A and B are benzene rings, A and B together form a fused ring, which is selected from the group consisting of carbazole, dibenzothiophene, and dibenzofuran.

9. The anti-blue light compound according to claim 1,
the compound is

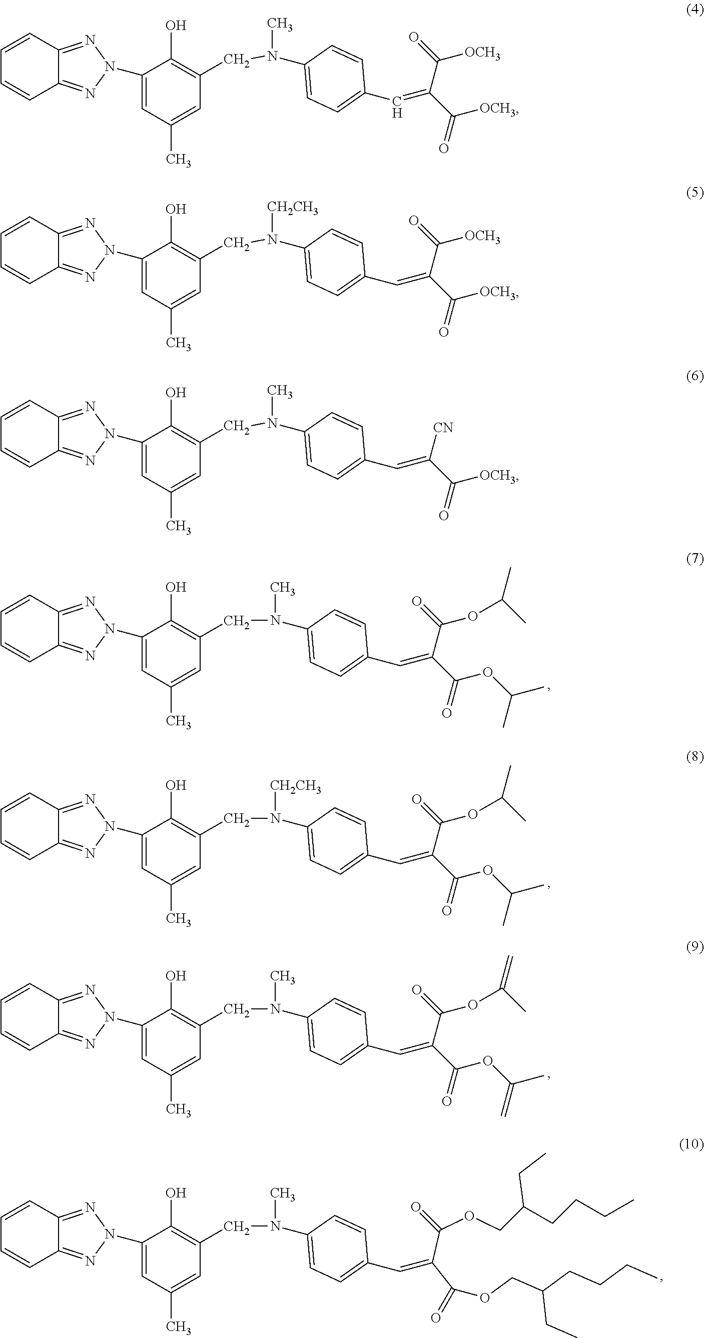

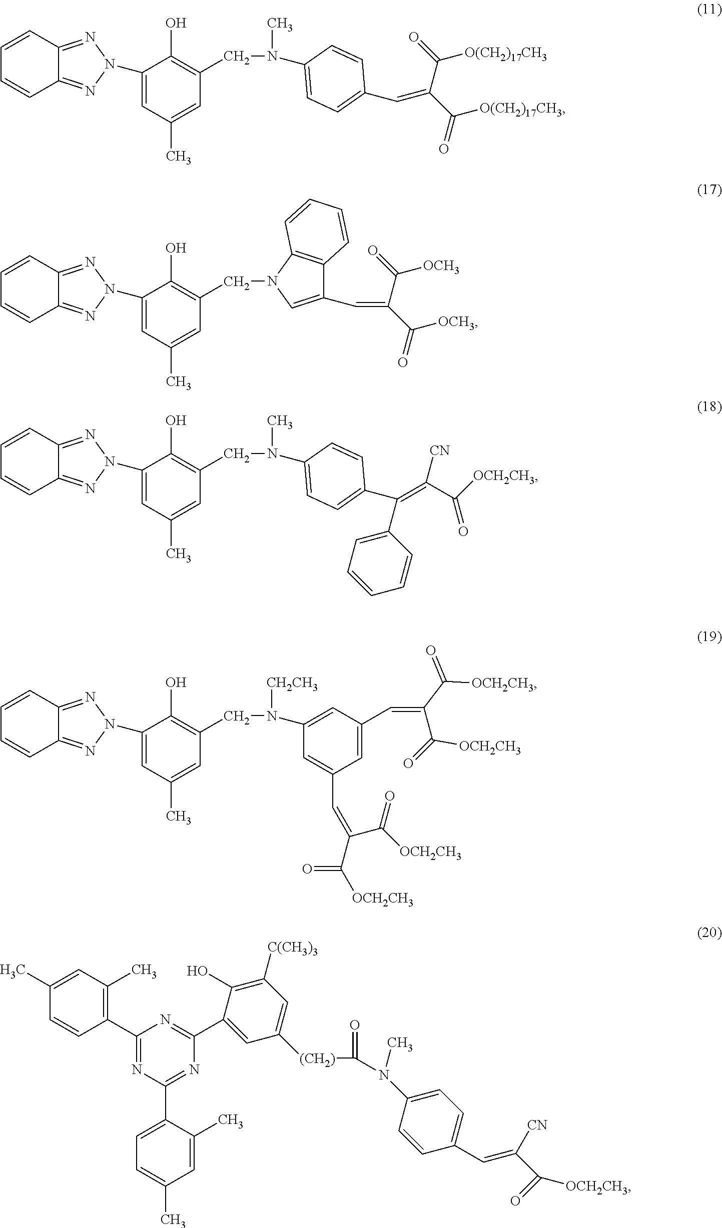

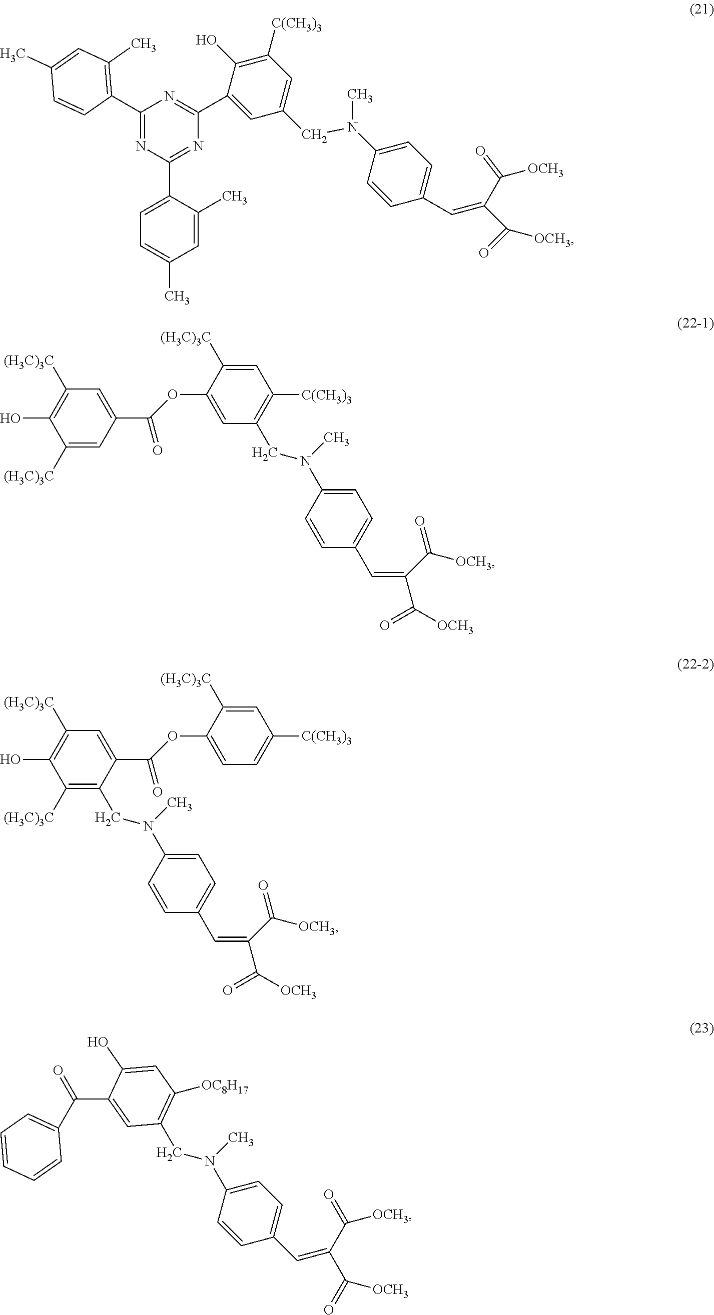

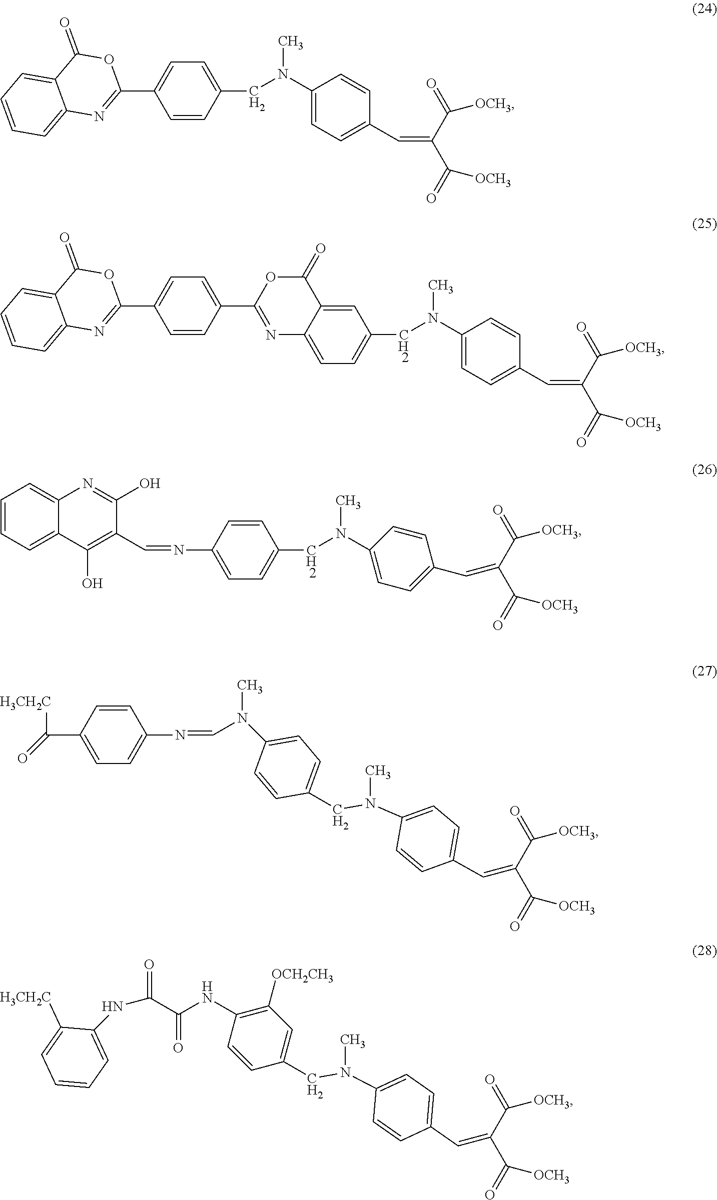

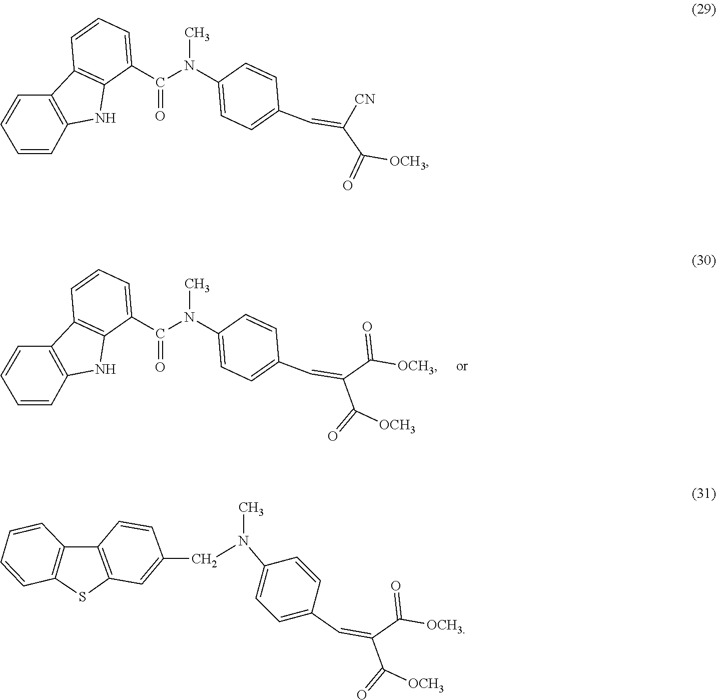

10. A method for manufacturing the anti-blue light compound according to claim 1, wherein, comprising the following step:
bonding covalently the anti-blue light compound and a UV blocking compound.

11. The method according to claim 10, comprises following reaction step:
A-R₁—B—R₂—C—CHO+Z--[—X]ₙ→formula (1) compound
or
A-R₁—B—CH₂Cl+NHR₁₅—C [symbol] -R₃---Z--[—X]ₙ]ₘ→formula(1)compound
wherein, A˜C, R₁˜R₃, X, Z, m, n, and the compound of formula (1) are the same as defined in claim 1;
R₁₅ is H, a linear or branched C₁˜C₈ alkyl or a phenyl.

12. An UV or/and blue light blocking composition, comprising a compound of formula (1) according to claim 1.
